(19) United States
(12) Patent Application Publication
(10) Pub. No.: US 2008/0281531 A1
(43) Pub. Date: Nov. 13, 2008

(54) Method for Diagnosing Depression

(76) Inventors: Kazuhito Rokutan, Osaka (JP); Tetsuro Ohmori, Tokushima (JP); Toshiro Saito, Hitachinaka (JP); Masayuki Ohta, Kodaira (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/048,241

(22) Filed: Mar. 14, 2008

(30) Foreign Application Priority Data
Mar. 15, 2007 (JP) 2007-065993

(51) Int. Cl. C12Q 1/68; G01N 33/50

(52) U.S. Cl. 702/20; 435/6

(57) ABSTRACT

This invention relates to a method for diagnosing whether or not a subject suffers from depression in a simple manner with high accuracy using the peripheral whole blood sample of the subject. Specifically, the present invention relates to a method for diagnosing depression comprising the steps of: measuring expression levels of 18 genes selected from the group consisting of FASLG; CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5, in peripheral blood isolated from a subject; and determining whether or not the subject suffers from depression based on the expression levels of the 18 genes.

### CLAIM OF PRIORITY

The present application claims priority from Japanese application JP 2007-065993 filed on Mar. 15, 2007, the content of which is hereby incorporated by reference into this application.

### BACKGROUND OF THE INVENTION

1. Field of the Invention

The present invention relates to a method of diagnosing depression. More particularly, the present invention relates to a method of diagnosing depression for determining whether or not a subject suffers from depression based on expression levels of specified 18 genes in the peripheral blood isolated from the subject.

2. Background Art

Depression is a disease with high lifetime morbidity of approximately 10%, and this rate is predicted to further increase in the future due to stress in contemporary society. This disease seriously afflicts patients mentally and physically and imposes enormous damage upon their social lives. In addition, it is a serious disease that often leads to suicide. It is deduced that many of the people who commit suicide (as many as 30,000 or more per year in Japan) are afflicted with depression. This disease is also deeply associated with societal problems such as truancy, unemployment, and social withdrawal or medical problems such as alcohol-related disorders. Establishment of methods of precisely diagnosing and promptly treating this disease is indispensable for improving the quality of life, and thus is an urgent need of society as a whole.

Diagnosis of depression is, however, far from simple. Cardinal symptoms of depression are, for example, depressive mood, hypobulia, loss of interest and pleasure, disrupted concentration and attention, lowered self-esteem and self-confidence, feelings of guilt and worthlessness, pessimism about the future, thoughts of suicide, sleep disorders, and loss of appetite. These symptoms have features peculiar to depression, which differ from depressed feelings experienced by anyone, and also differ from the lowered mental activity and sense of exhaustion experienced by people afflicted with physical diseases. The symptoms of depression are mainly comprehended by taking a precise medical history, questioning when and how the symptoms in terms of mental activity were developed and what types of damages have been imposed upon their social and domestic lives, and confirming various symptoms based on a patient's attitude or the contents of conversations during consultation. For example, family medical history, anamnesis, physical conditions, early developmental history, life history, personality inclination, premorbid social adaptation, and the occurrence of any episode(s) that had triggered the disease can be important references. In order to accurately comprehend these factors, an interview needs to be conducted by a highly skilled specialist in psychiatric medicine for approximately 1 hour. Further, it should be confirmed that a patient does not have any major abnormalities in terms of general physical or neurological conditions. If necessary, the possibility of the existence of organic brain disorders is to be eliminated by electroencephalography or brain imaging tests. The patient is then subjected to diagnosis. The findings are compared with the diagnostic standards issued by the World Health Organization (WHO) or the American Psychiatric Association, and the diagnosis can be generally confirmed.

As a major drawback, conventional diagnostic methods require skilled techniques. Needless to say, thorough knowledge and practice concerning depression are required. However, there are numerous psychological, mental, and physical states that result in the exhibition of depressive conditions even though they are not forms of depression. Differential diagnosis also becomes essential. Accordingly, diagnosis must be conducted by a thoroughly trained specialist in psychiatric medicine. Depression, which is a common disease with lifetime morbidity of approximately 10%, however, is often the subject of consultation with primary care doctors. Diagnosis of depression without objective medical findings is not always easy for general doctors who may not be acquainted with psychiatric consultation. Depression is a medical disease that requires treatment of the body (brain), including medication. Accordingly, it is difficult for specialists in clinical psychology, such as clinical psychotherapists, or mental health workers, such as public health nurses, to independently diagnose depression.

Technical skill is required for diagnosis mainly because of a lack of simple and objective methods of diagnosis regarding symptoms. Although there is a screening method utilizing a self-administered questionnaire at present, people tend to fill in the questionnaire based on their subjective viewpoints. Thus, genuine depression cannot be distinguished from depressed feelings caused by personality-based factors, environmental factors, or poor physical conditions. Symptom rating scales employed by doctors are often used in determination of severity, although adequate questioning is required to evaluate each item. Thus, such methods cannot be alternatives to diagnosis.

Many testing methods have been heretofore attempted, with the aim of utilizing them as objective indicators. Depression causes functional alteration in brain monoamine systems. This alteration is known to have a considerable influence upon the neuroendocrine system, the neuroimmune system, and the autonomic nervous system via psychosomatic correlation. In particular, the application of the results of a dexamethasone suppression test that allows accurate comprehension of neuroendocrine abnormalities, i.e., a minor level of adrenal cortical hormone hypersecretion, to diagnosis of depression has been extensively examined from the 1980s onwards. Clinical application thereof was, however, not realized due to the necessity for complicated procedures such as the administration of test drugs and limitations in terms of sensitivity or specificity. At the study phase, other abnormalities in the neuroendocrine system, the neuroimmune system, the autonomic nervous system, circadian rhythms, sleep architecture, and the like had been reported. Recently, changes regarding conditions of brain blood flow or brain monoamine receptors are also pointed out as objective indicators, although they are still disadvantageous in terms of sensitivity and reproducibility. Given the aforementioned factors, diagnosis of a complicated psychiatric disease, i.e., depression, is difficult by a method of testing limited factors. Enormous amounts of time and labor are required to perform conventional testing methods and to diagnose the disease. From the viewpoint of simplicity, conventional techniques cannot be applied to routine medical care at present.

The present inventors analyzed the expression patterns peculiar to patients afflicted with depression via peripheral-blood-targeted gene expression analysis. They developed a method for diagnosing depression using such feature as an indicator and reported such method (JP Patent Publication No. 2004-208547A (U.S. Patent Publication No. 2004-185474) and JP Patent Publication No. 2005-312435A (U.S. Patent Publication No. 2005-239110)). The method disclosed therein, however, involves the use of microarrays having about 1,500 genes mounted thereon to search for disease-associated genes, and such number of genes is very small compared with the types of gene transcripts that are expressed in peripheral blood cells (i.e., about 10,000 to 20,000 types). Accordingly, such a search of markers may not be sufficient, and marker genes that exhibit expression behaviors more peculiar to patients afflicted with depression may be missed.

### SUMMARY OF THE INVENTION

An object of the present invention is to provide a simple and accurate method for diagnosing depression via assay of a large number of factors. More particularly, microarrays that are capable of assay of expression levels of as many as 41,000 types of gene transcripts, which represents the entire number of human genes, at one time are used to select marker genes that exhibit expression levels peculiar to patients afflicted with depression.

In the past, the catecholamine hypothesis and the indoleamine hypothesis were proposed as causes of depression. In addition, the GABA hypothesis, the glutamine hypothesis, the dopamine hypothesis, the neurogenesis hypothesis, and the like have been proposed as causes of depression in recent years. Many discrepancies in these hypotheses have been pointed out, and they have not yet resulted in conclusions. Linkage studies and association studies based on molecular genetic engineering and the search for sensitive chromosome domains by linkage analysis have been carried out. In the case of a disease such as depression, the diathesis (biological feature) thereof is generated through interactions among multiple genes and environmental factors such as stress, and therefore pathogenic gene analysis is extremely difficult. Based on past gene analysis, genes such as those related to serotonin transporter, serotonin 1A/2C receptor, dopamine D2/D3 receptor, dopamine transporter, tyrosine hydroxylase, tryptophan hydroxylase, and monoamine oxidase have been reported as candidate functional genes associated with depression. Some researchers are, however, skeptical about the aforementioned reports, and additional tests have been conducted thereon.

The present inventors have focused on peripheral leukocytes that can be easily obtained as specimens and express many receptors of stress-associated factors in order to objectively diagnose the conditions of depression, which is often caused by stress. They extracted RNAs directly from the whole blood because isolation of leukocytes from the whole blood would impose serious damage to such leukocytes. They extensively analyzed the expression patterns of genes exhibiting high expression levels in leukocytes and then patterned the expression levels. Through this analysis, microarrays that are capable of extensive measurement of as many as 41,000 types of gene transcripts, which represents the entire number of human genes, are used to search for and select marker genes that exhibit expression levels peculiar to patients afflicted with depression.

Thus, the present inventors determined 18 novel genes that can serve as marker genes of depression and completed development of a method for diagnosing the morbidity of a subject with depression with high accuracy based on the expression levels of such 18 genes or mean expression level thereof.

The present invention provides a method for diagnosing depression comprising the steps of: measuring expression levels of 18 genes selected from the group consisting of FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5, in peripheral blood isolated from a subject; and determining whether or not the subject suffers from depression based on the expression levels of the 18 genes.

According to an embodiment, the expression levels of the 18 genes of the subject are compared with the expression levels of the same genes of healthy individual to determine whether or not the subject suffers from depression.

According to another embodiment, the expression ratio of the expression levels of the 18 genes of the subject to the expression levels of the same genes of healthy individual is obtained, and the expression ratio is compared with predetermined date concerning expression levels of the 18 genes of depressed patient and expression levels of the 18 genes of healthy individual to determine whether or not the subject suffers from depression. This comparative analysis can be carried out using, for example, a support vector machine.

According to another embodiment, a mean value of expression levels of the 18 genes of the subject is obtained and the mean value is compared with a mean value of expression levels of the 18 genes of healthy individual to determine whether or not the subject suffers from depression. When the mean value of the 18 genes of the subject is significantly lower than that of healthy individual, specifically, the subject can be determined as having a high possibility of suffering from depression.

Gene expression levels can be measured in a simple manner with the use of nucleic acid-immobilized solid substrates, such as DNA chips or arrays.

Expression levels of some of the aforementioned 18 genes may be used to determine whether or not the subject suffers from depression. Another aspect of the present invention provides a method for diagnosing depression, wherein the expression level of at least one of the aforementioned 18 genes is measured to determine whether or not the subject suffers from depression based on the expression level.

In this method, the expression level of at least one of the aforementioned 18 genes of the subject is compared with a predetermined reference value to determine whether or not the subject suffers from depression based on the comparison results.

Alternatively, a ratio of the expression level of at least one of the aforementioned 18 genes to the predetermined reference value is obtained, and the ratio is compared with a predetermined threshold value to determine whether or not the subject suffers from depression based on the comparison results.

Also, the mean value of expression levels of at least two of the aforementioned 18 genes of the subject is obtained, and the mean value is compared with a predetermined threshold value to determine whether or not the subject suffers from depression based on the comparison results. When the mean value of expression levels of at least two of the aforementioned 18 genes of the subject is lower than the aforementioned threshold value, for example, the subject can be determined as having a high possibility of suffering from depression.

The present invention also provides a program for performing the method for diagnosing depression of the present invention. The program of the present invention comprises:

1) a means for inputting the expression levels of 18 genes selected from the group consisting of FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5, in the peripheral blood isolated from a subject;

2) a means for storing data concerning expression levels of the aforementioned 18 genes of depressed patients and of healthy individual that have been inputted in advance;

3) a means for comparing the expression levels of the 18 genes of the subject with expression levels of the 18 genes of healthy individual;

4) a means for determining whether or not the subject suffers from depression based on the results of comparison; and

5) a means for outputting the results of determination.

According to one embodiment, in 3) above, an expression ratio of the expression levels of the 18 genes of the subject to those of healthy individual is obtained in order to compare said expression ratio with an expression ratio of expression levels of the 18 genes of depressed patient to expression levels of the 18 genes of healthy individual that have been stored in advance. This analysis can be carried out using, for example, a support vector machine.

According to another embodiment, in 3) above, the mean value of expression levels of the 18 genes of the subject is obtained, and this mean value is compared with the mean value of expression levels of the 18 genes of healthy individual.

The program of the present invention preferably comprises a means for storing the expression levels of the 18 genes of the subject and updating the data of the depressed patient and that of healthy individual according to need, in addition to the aforementioned means.

FIG. 1 schematically shows the method for diagnosis of depression of the present invention. In the present invention, peripheral blood is collected from a subject, RNA is extracted, and its expression profile is examined, thereby resulting in diagnosis of whether or not the subject suffers from depression. Approximately 2 to 5 ml of peripheral blood is sufficient for diagnosis.

Techniques for examining the gene expression levels employed in the present invention are not limited to nucleic acid-immobilized solid substrates such as a DNA chip or a microarray. For example, the availability of techniques such as quantitative PCR or Northern blotting is apparent for those skilled in the art.

According to the present invention, preferably, the expression data of the patients afflicted with depression and of healthy individuals are stored in the database in combination with the clinical information, and the expression data of the subject is analyzed with reference to the database to examine the condition of depression of the subject. It is apparent that a means for data analysis is not limited to a support vector machine, and the algorithm of other learning machines can also be used. Preferably, expression data for patients afflicted with depression and those for healthy individuals are previously stored in the computer, and the computer is allowed to determine which of the expression patterns for patients or healthy individuals are more similar to the subject's expression patterns, thereby diagnosing the conditions of depression in the subject. FIG. 2 schematically shows the system for diagnosing depression.

The publications (JP Patent Publication No. 2004-208547A (U.S. Patent Publication No. 2004-185474) and JP Patent Publication No. 2005-312435A (U.S. Patent Publication No. 2005-239110)) each disclose a method for diagnosing whether or not a subject suffers from a disease by a method of hierarchical cluster analysis, wherein the expression levels of marker genes are used as indicators. According to cluster analysis, a dendrogram for classification of the subjects' expression patterns into the patient group or healthy individual group must be selected by a human in the last stage. Thus, it is difficult to maintain objectivity. According to a classification method using a support vector machine, however, selection is made by a computer, and objectivity is thus maintained (Proceedings of National Academy of Sciences of the United States of America, Vol. 97, Issue 1, 262-267, (2000), “Knowledge-based analysis of microarray gene expression data using support vector machines”). Further, distance from a borderline (i.e., the borderline between patients afflicted with depression and healthy individuals) can also be determined, and the distance of the subject from the borderline can be objectively determined. Accordingly, such technique is suitable for a method of the present invention, wherein the expression levels of a plurality of genes are comprehensively measured to evaluate the affliction.

The present invention provides a method of diagnosing depression by collectively quantifying the RNA expression levels in peripheral leukocyte blood to find evidence that is peculiar to depression. This would innovatively improve medical care for depression.

The method of the present invention can conduct the analysis with the use of 2 to 5 ml of blood obtained by conventional blood sampling without special cooperation provided by a patient. This diagnostic method can be carried out in a non-invasive, simple, and routine manner. This method of multidimensionally comprehending biological functions based on numerous RNA expression levels is more adequate as a method of diagnosing complicated psychiatric diseases involving both mental and physical conditions such as depression in terms of its principle compared with the conventional method that assays only limited factors.

The results attained by the method of the present invention can be simply and clearly evaluated, they can be easily employed by primary care doctors as objective indicators for depression, and they are extremely useful for the establishment of diagnosis and introduction of therapy. A high-risk group can be selected from among the groups of people in a simple, accurate, and cost-effective manner through medical checkups or complete physical examinations provided by workplaces, schools, and communities. This enables early detection of depression. Accordingly, the method of the present invention significantly contributes to the improvement of peoples' mental health from the viewpoint of preventive care.

The usefulness of the method according to the present invention is not limited to primary care and medical checkups. Specialists in psychiatric medicine can apply this technique to the search for psychological, social, and environmental factors associated with the development of depression, evaluation of clinical conditions, diagnosis, evaluation of treatment, and determination of prognosis. Thus, this technique can be a revolutionary test technique in the field of psychiatric medicine.

### BRIEF DESCRIPTION OF THE DRAWINGS

FIG. 1 schematically shows the method of diagnosing depression according to the present invention.

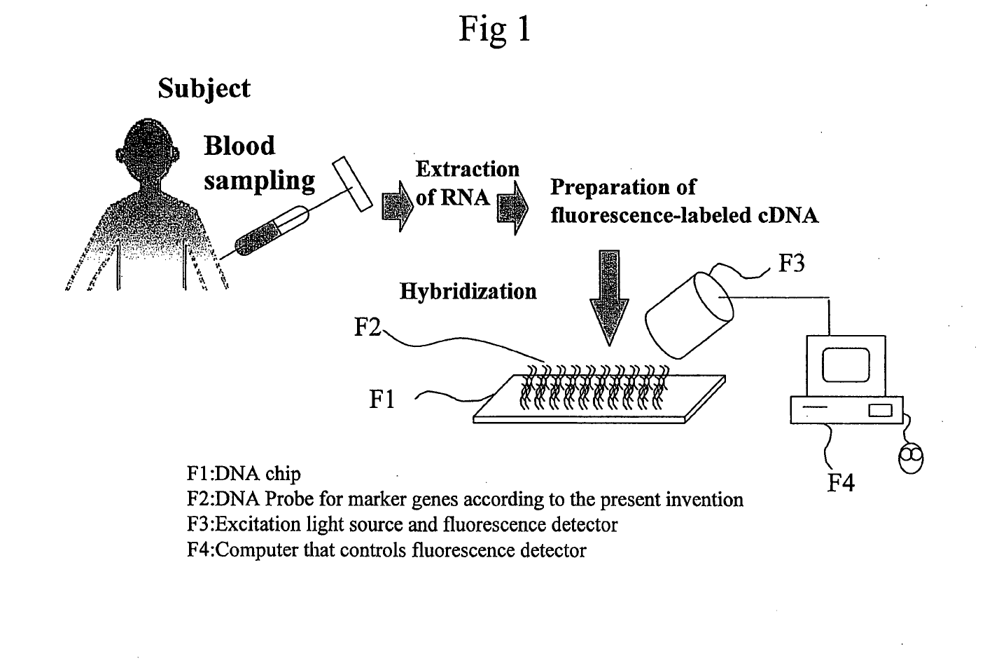

FIG. 2 schematically shows the system of diagnosing depression according to the present invention.

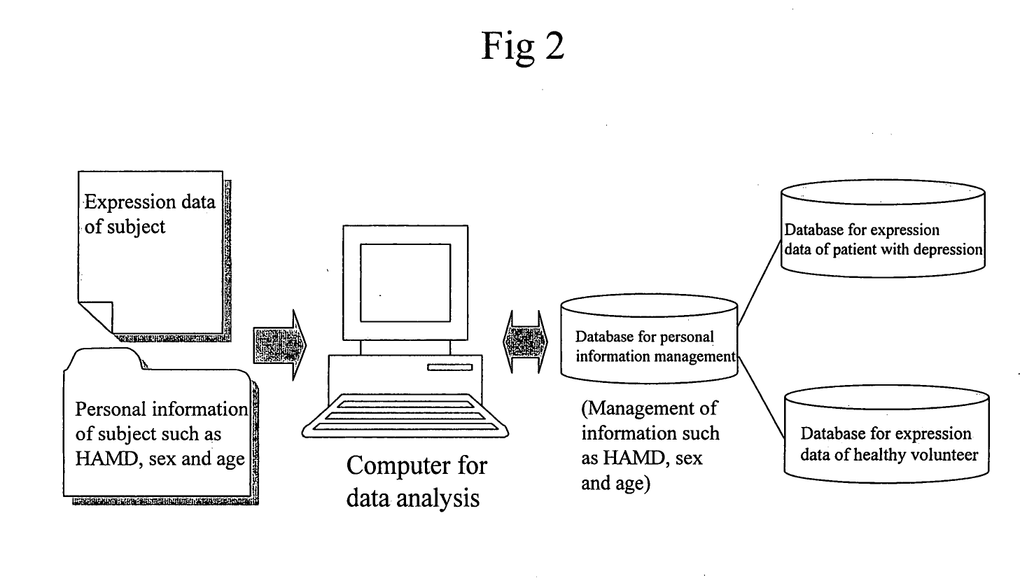

FIG. 3 shows the plot of p-values obtained via a significant difference test based on the expression levels of genes selected from 631 genes in ascending order of p-values.

FIG. 4 shows changes in mean logarithmic values for the expression levels of 18 genes (i.e., the expression ratio in relation to the reference dataset) with the elapse of treatment time.

FIG. 5 shows comparison of the mean expression level of 18 genes between patients afflicted with depression (D1) and healthy individuals (N1) (i.e., the mean Log ratio).

### DETAILED DESCRIPTION OF THE PREFERRED EMBODIMENTS

Hereafter, the embodiments of the present invention are described in detail with reference to concrete examples.

### Example 1

The methods for searching for and selecting the marker genes according to the present invention are described.

The present inventors collected blood from patients and healthy individuals as described below. RNA was extracted from the whole blood, and gene expression of patients was then analyzed using DNA chips, along with that of healthy individuals. A DNA chip comprises DNA fragments having nucleotide sequences corresponding to numerous genes immobilized on a substrate such as a glass substrate, and it is used for detecting DNA or RNA in a sample by hybridization.

Target patients were as follows. Target patients were those who had agreed with the written description for participating in the research for developing the present diagnostic method selected from among untreated patients afflicted with depression who had visited the Department of Psychiatry and Neurology of the Tokushima University Hospital between November 2002 and December 2006. This research was approved by the ethics committee of Tokushima University Hospital. Diagnosis was made in accordance with depressive episode specified in the International Classification of Diseases, 10th revision (ICD-10). Patients with serious physical complications or those taking therapeutic agents for physical diseases were excluded. Blood was collected by a doctor or nurse between 10:00 am and 1:00 pm from the patients under fasting conditions through cubitus veins under resting conditions.

Detailed information of subjects is summarized in Table 1. Forty six patients whose samples before treatment had been obtained were 17 males and 29 females aged 17 to 76 (42 years old on average), and their Hamilton scores were 19.8 on average (standard deviation: 6.8).

TABLE 1
Information of subjects

| | Number of | | | | | | | |
|---|---|---|---|---|---|---|---|---|
| | individuals | | | Age | | | HAM-D | |
| Group | Total | Male | Female | Mean | Youngest | Oldest | Mean | S.D. |
| Patients afflicted with depression (before | 46 | 17 | 29 | 41.9 | 17 | 76 | 19.8 | 6.8 |
| treatment) | | | | | | | | |
| Patients afflicted with depression (1 month | 32 | 14 | 18 | 41.9 | 21 | 72 | 7.8 | 6.4 |
| after initiation of treatment) | | | | | | | | |
| Patients afflicted with depression (2 months | 7 | 6 | 1 | 37.9 | 21 | 68 | 5.6 | 6.5 |
| after initiation of treatment) | | | | | | | | |
| Patients afflicted with depression (3 months | 1 | 1 | 0 | 30.0 | 30 | 30 | 4.0 | |
| after initiation of treatment) | | | | | | | | |
| Healthy individuals | 122 | 49 | 73 | 45.1 | 21 | 88 | | |

A hundred and twenty two healthy individuals whose samples had been obtained were 49 males and 73 females aged 21 to 88 (45 years old on average) (i.e., control subjects. The male-female ratio and the age of patients before treatment were substantially the same as those of healthy individuals. Blood was collected from healthy individuals under fasting conditions between 10:00 am and 1:00 pm.

Samples after antidepressant treatment were obtained from 40 patients, and those samples were obtained one month after the initiation of treatment (32 patients), two months after the initiation of treatment (7 patients), and three months after the initiation of treatment (1 patient). The Hamilton scores after treatment were 19.8 points, 7.8 points, and 4 points on average, respectively. A total of 208 specimens; i.e., 46 specimens obtained from patients afflicted with depression before treatment, 40 specimens obtained from patients afflicted with depression after treatment, and 122 specimens obtained from healthy individuals, were subjected to analysis.

Blood (5 ml; 2.5 ml×2) was collected from all subjects using a PAXgene Blood RNA System (Qiagen), and total RNA was extracted. The yield of total RNA was 5 μg to 15 μg. Subsequently, quality of total RNA extracted from the subjects was inspected using the Bioanalyzer 2100 (Agilent) to confirm that total RNA had not been decomposed. Total RNA (0.2 μg) was then subjected to the in vitro transcription reaction to synthesize cRNA into which aminoallyl-CTP had been introduced in the presence of aminoallyl-CTP using the Agilent reagent that amplifies and synthesizes cRNA (Low RNA Input Linear Amp Kit PLUS, One-Color). Subsequently, an amino group in the synthesized cRNA was subjected to coupling to a succinimide-containing fluorescent dye (Cy3, Amersham) to synthesize fluorescent-labeled RNA. Subsequently, the fluorescent-labeled RNA was subjected to hybridization to the Agilent microarrays (Whole Human Genome Microarray 4 Pack) at 65° C. for 17 hours. The microarrays were washed in accordance with the Agilent's given protocols, and the fluorescent image was read using the Agilent scanner (Agilent). The image data was converted into the numerical data using a special software, Feature Extraction (Agilent). Subsequently, normalization was carried out so that a sum of signal intensities of genes exhibiting signal intensities of 25% to 0.75% would be the same among 208 specimens. The values representing the same sums were 11, 234, and 345. Subsequently, genes exhibiting 100 or higher signal intensities in 104 or more specimens, i.e., a half of the entire dataset comprising 208 specimens, were extracted, these genes were designated as genes expressed in the peripheral blood, and this group of target genes was subjected to the following analysis. As target genes, 21,895 genes were extracted.

After normalization and gene selection in accordance with signal intensities, the data of 122 specimens obtained from healthy individuals was subjected to determination of the mean expression intensity of the genes to prepare a dataset of mean values as the reference dataset. Subsequently, the data of expression intensity of 208 specimens each was divided by the reference dataset to determine the expression intensity ratios among the 208 specimens. The data of expression ratios among the 208 specimens was subjected to the following data analysis.

In order to extract genes that exhibit significantly different expression levels between the group of 46 specimens obtained from patients before treatment and the group of 122 specimens obtained from healthy individuals, a t-test was carried out at the 0.05 significance level with Bonferroni correction for multiple comparison without the homoscedasticity assumption. As a result, 631 genes were extracted. According to the expression levels, three-fourth or more of the genes exhibited the decrease in the expression levels in patients than in healthy individuals, and the degrees of decrease were substantially uniform.

Thus, these 631 genes were found to exhibit sufficient expression levels in the peripheral blood samples. Also, these genes were found to be useful as marker genes exhibiting significant differences in expression levels between healthy individuals and patients.

### Example 2

Erythrocytes do not contain nuclei. Thus, most RNAs in the whole blood samples are considered to originate from leukocytes. Leukocytes consist of monocytes, granulocytes, and lymphocytes, and, for example, T cells, B cells, and NK cells are also lymphocytes. Accordingly, the whole blood samples used in this test contain RNAs derived from a numerous different kinds of cells. Thus, the gene expression levels examined in this test are the result of multiplying the gene expression levels in each type of cells by the number of the cell. If the numbers of specific types of cells in the blood are different between patients and healthy individuals, accordingly, a significant difference would be observed in the expression levels of genes exhibiting specifically high expression levels in the cells. The differences in expression levels of such genes result from differences in the number of the cells; in other words, the differences in gene expression levels would be substantially uniform.

The cells from which the 631 genes exhibiting significantly different levels of expression between the group of healthy individuals and the group of patients described in Example 1 mainly originate were inspected in the following manner.

About 30 ml of blood was collected from two healthy individuals, T cells, neutrophil leucocytes, and monocytes were fractionated from 25 ml thereof using surface antigen-recognizing micro-magnetic beads (Miltenyi Biotec K. K.), RNAs were extracted therefrom, and RNA was extracted from 5 ml of the remaining whole blood. Gene expression was compared and analyzed between the whole blood and each fraction. The expression level determined as the mean between two subjects was used to extract genes that are expressed specifically for each of T cells, neutrophil leucocytes, and monocytes in terms of higher expression levels by two times or more in comparison with the other two fractions and differences in expression levels by two times or less between two subjects. As a result, 141 genes, 120 genes, and 204 genes that are expressed specifically for T cells, neutrophil leucocytes, and monocytes, respectively, were extracted. In order to inspect the correlation between these genes and the 631 genes extracted by the intergroup test, the data of the 208 specimens was subjected to cluster analysis for grouping the target 21,895 genes, and the dataset of the 631 genes and that of the genes expressed specifically for each fraction were mapped thereon. As a result, the target 21,895 genes were roughly divided into four groups, and 281 of 631 depression-associated genes were found to be collectively present in the group of genes collectively comprising 107 of the 141 genes expressed specifically for T-cells. Genes expressed specifically for other fractions were collectively present in the other group of genes. This indicates that 631 depression-associated genes exhibiting significantly different expression levels between the group of healthy individuals and the group of patients originate from T cells. Affliction with depression causes changes in the number of T cells or the abundance of a variety of T cells, such as killer T cells, helper T cells, and suppressor T cells.

The above results demonstrate that depression can be diagnosed by analyzing expression of genes obtained from the peripheral whole blood of subjects, measuring the expression levels of genes exhibiting the expression levels at least two times higher in T cells than in other blood cells, and determining whether or not a subject suffers from depression based on the results of the expression levels of the genes.

### Example 3

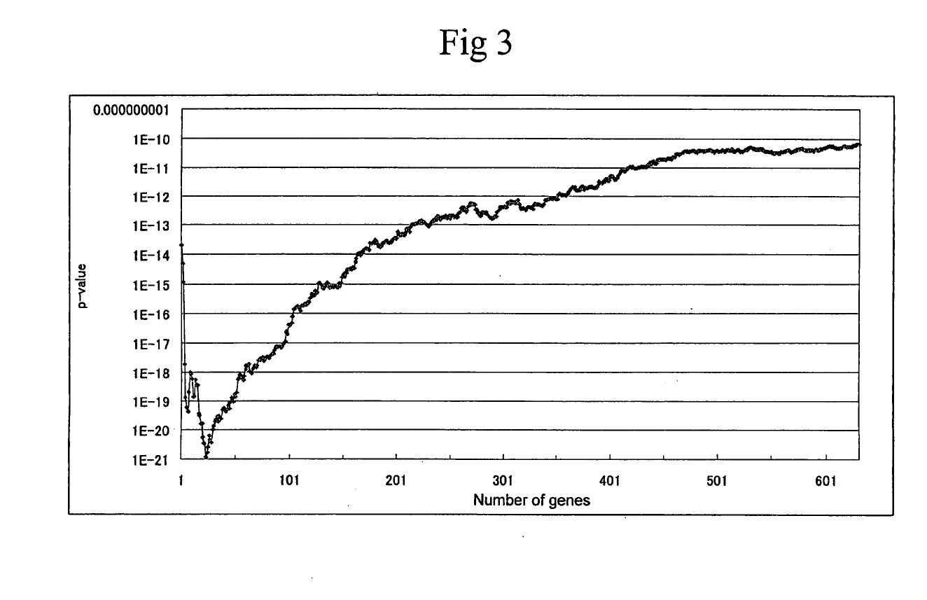

Among the 631 depression-associated genes described in Example 1, the group of genes that should be focused in order to effectively diagnose depression was examined. Genes were selected from among the 631 genes in ascending order of p values, the mean expression levels thereof were calculated, and the determined mean was subjected to a significant difference test between the group of 122 healthy individuals and the group of 46 patients to inspect the resulting p values. Specifically, a significant difference test was carried out using the mean Log value among genes for the ratio of the expression levels of patients' genes to the expression levels of healthy individuals' genes. FIG. 3 shows the results thereof. As the number of target genes for determining the mean used for diagnosis increases, the resulting p values (obtained by the intergroup test using the mean) decrease. If genes exhibiting large p values (the value obtained from only one gene) are targeted, however, the resulting p values (the means of the groups of target genes) were found to increase gradually. This demonstrated the presence of a set of genes exhibiting the lowest p value. Table 2 shows the set of genes. The p values shown in Table 2 were determined when extracting 631 genes in Example 1, which had been subjected to multiple correction.

TABLE 2
List of 18 genes for distinguishing depressed patient from healthy individual

| | Target | Target | | | |
|---|---|---|---|---|---|
| p-value | Accession | Symbol | Target Description | UniGene | GeneID |
| 4.36E−10 | NM_000639 | FASLG | Homo sapiens Fas ligand (TNF superfamily, member 6) (FASLG), mRNA | Hs.2007 | 356 |
| | | | [NM_000639] | | |
| 5.95E−09 | NM_001337 | CX3CR1 | Homo sapiens chemokine (C-X3-C motif) receptor 1 (CX3CR1), mRNA | Hs.78913 | 1524 |
| | | | [NM_001337] | | |
| 3.83E−08 | NM_013351 | TBX21 | Homo sapiens T-box 21 (TBX21), mRNA [NM_013351] | Hs.272409 | 30009 |
| 4.44E−08 | NM_002166 | ID2 | Homo sapiens inhibitor of DNA binding 2, dominant negative helix-loop-helix | Hs.180919 | 3398 |
| | | | protein (ID2), mRNA [NM_002166] | | |
| 9.78E−08 | NM_021181 | SLAMF7 | Homo sapiens SLAM family member 7 (SLAMF7), mRNA [NM_021181] | Hs.517265 | 57823 |
| 1.37E−07 | NM_007173 | PRSS23 | Homo sapiens protease, serine, 23 (PRSS23), mRNA [NM_007173] | Hs.25338 | 11098 |
| 2.34E−07 | NM_006826 | YWHAQ | Homo sapiens tyrosine 3-monooxygenase/tryptophan 5-monooxygenase | Hs.74405 | 10971 |
| | | | activation protein, theta polypeptide (YWHAQ), mRNA [NM_006826] | | |
| 2.67E−07 | NM_007375 | TARDBP | Homo sapiens TAR DNA binding protein (TARDBP), mRNA [NM_007375] | Hs.300624 | 23435 |
| 2.70E−07 | NM_000024 | ADRB2 | Homo sapiens adrenergic, beta-2-, receptor, surface (ADRB2), mRNA | Hs.591251 | 154 |
| | | | [NM_000024] | | |
| 3.40E−07 | NM_138558 | PPP1R8 | Homo sapiens protein phosphatase 1, regulatory (inhibitor) subunit 8 (PPP1R8), | Hs.533474 | 5511 |
| | | | transcript variant 2, mRNA [NM_138558] | | |
| 5.24E−07 | NM_172250 | MMAA | Homo sapiens methylmalonic aciduria (cobalamin deficiency) cblA type (MMAA), | Hs.452864 | 166785 |
| | | | mRNA [NM_172250] | | |
| 6.23E−07 | NM_003129 | SQLE | Homo sapiens squalene epoxidase (SQLE), mRNA [NM_003129] | Hs.71465 | 6713 |
| 1.09E−06 | NM_000284 | PDHA1 | Homo sapiens pyruvate dehydrogenase (lipoamide) alpha 1 (PDHA1), mRNA | Hs.530331 | 5160 |
| | | | [NM_000284] | | |
| 1.24E−06 | NM_032782 | HAVCR2 | Homo sapiens hepatitis A virus cellular receptor 2 (HAVCR2), mRNA [NM_032782] | Hs.616365 | 84868 |
| 1.24E−06 | NM_013277 | RACGAP1 | Homo sapiens Rac GTPase activating protein 1 (RACGAP1), mRNA [NM_013277] | Hs.505469 | 29127 |
| 1.97E−06 | NM_001620 | AHNAK | Homo sapiens AHNAK nucleoprotein (desmoyokin) (AHNAK), transcript variant 1, | Hs.502756 | 79026 |
| | | | mRNA [NM_001620] | | |
| 3.11E−06 | NM_030760 | EDG8 | Homo sapiens endothelial differentiation, sphingolipid G-protein-coupled receptor, | Hs.501561 | 53637 |
| | | | 8 (EDG8), mRNA [NM_030760] | | |
| 3.24E−06 | NM_004419 | DUSP5 | Homo sapiens dual specificity phosphatase 5 (DUSP5), mRNA [NM_004419] | Hs.2128 | 1847 |

Tables 3 to 6 each show the data of expression levels (fluorescent intensities) of 18 genes of patients, and Tables 7 to 17 each show the data of expression levels (fluorescent intensities) of 18 genes of healthy individuals. A comparison of the data of patients with the data of healthy individuals shows significantly lowered expression levels of the 18 genes of patients afflicted with depression compared with those of healthy individuals.

TABLE 3

| | Patient | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| | Age | | | | | | | | | | | |
| | 68 | 43 | 23 | 64 | 30 | 67 | 30 | 57 | 26 | 37 | 28 | 55 |
| Gene | Sex | | | | | | | | | | | |
| symbol | Male | Female | Female | Male | Male | Female | Female | Female | Female | Male | Male | Female |
| FASLG | 608.3 | 808.6 | 356.1 | 525.3 | 337.9 | 674.0 | 662.7 | 519.2 | 650.4 | 378.9 | 541.8 | 828.6 |
| CX3CR1 | 36728.0 | 21857.3 | 14531.6 | 29285.7 | 19216.1 | 25079.5 | 24453.4 | 21836.4 | 31783.6 | 27270.5 | 12788.2 | 22773.5 |
| TBX21 | 11742.9 | 9943.5 | 3377.5 | 10459.9 | 5982.6 | 6323.9 | 7551.1 | 7070.6 | 11310.8 | 5438.5 | 4526.0 | 6531.0 |
| ID2 | 4498.7 | 3425.3 | 3390.9 | 5449.9 | 2897.3 | 3284.8 | 4278.6 | 5081.4 | 4780.3 | 2349.7 | 3570.8 | 6546.5 |
| SLAMF7 | 3811.7 | 4491.6 | 2551.3 | 3651.6 | 1847.6 | 4307.0 | 3084.6 | 3461.0 | 3426.7 | 2060.0 | 2052.2 | 5197.4 |
| PRSS23 | 435.7 | 591.1 | 84.5 | 194.7 | 185.0 | 232.0 | 338.0 | 373.4 | 405.3 | 158.8 | 138.2 | 195.4 |
| YWHAQ | 14897.3 | 19594.3 | 10674.3 | 12112.4 | 12313.8 | 8681.7 | 15192.1 | 15061.0 | 13897.9 | 10624.5 | 13098.7 | 14974.6 |
| TARDBP | 13105.7 | 11152.5 | 11560.1 | 11321.8 | 9933.6 | 7847.1 | 11221.8 | 9183.2 | 12105.6 | 9656.6 | 10831.7 | 12211.6 |
| ADRB2 | 2882.6 | 3937.2 | 1449.3 | 2655.5 | 1969.0 | 1932.4 | 2465.8 | 2139.3 | 3395.9 | 1208.4 | 2565.3 | 2970.9 |
| PPP1R8 | 2544.3 | 2750.4 | 2480.9 | 2575.9 | 2495.4 | 2301.9 | 2600.7 | 2370.8 | 2360.0 | 1806.5 | 2719.4 | 3314.9 |
| MMAA | 183.4 | 220.2 | 173.7 | 170.6 | 152.3 | 207.5 | 214.4 | 187.7 | 184.8 | 151.5 | 234.3 | 250.5 |
| SQLE | 759.8 | 625.0 | 403.3 | 466.9 | 376.7 | 233.0 | 519.6 | 354.5 | 397.5 | 325.5 | 541.4 | 599.7 |
| PDHA1 | 4212.7 | 4507.7 | 3678.6 | 4464.6 | 3595.2 | 3279.4 | 4090.8 | 3487.0 | 4578.3 | 3398.7 | 4472.1 | 5252.1 |
| HAVCR2 | 1846.0 | 1950.1 | 1347.6 | 2428.6 | 1510.6 | 1342.4 | 1445.6 | 1249.3 | 1353.6 | 1527.0 | 1738.7 | 1757.7 |
| RACGAP1 | 625.7 | 433.7 | 288.9 | 312.2 | 310.6 | 258.4 | 378.5 | 372.9 | 398.8 | 279.1 | 329.3 | 371.8 |
| AHNAK | 11839.4 | 9486.1 | 9867.6 | 9349.4 | 10207.3 | 8281.9 | 10347.1 | 7711.0 | 10487.5 | 13114.4 | 9429.4 | 10447.7 |
| EDG8 | 527.4 | 505.0 | 100.8 | 83.4 | 213.0 | 193.4 | 274.4 | 83.6 | 157.4 | 223.1 | 217.0 | 319.4 |
| DUSP5 | 222.3 | 256.7 | 125.3 | 137.3 | 129.6 | 113.9 | 205.5 | 149.6 | 142.8 | 158.5 | 162.3 | 162.5 |

TABLE 4

| | Patient | | | | | | | | | | | |
|---|---|---|---|---|---|---|---|---|---|---|---|---|
| | 13 | 14 | 15 | 16 | 17 | 18 | 19 | 20 | 21 | 22 | 23 | 24 |
| | Age | | | | | | | | | | | |
| | 55 | 32 | 43 | 20 | 25 | 55 | 23 | 23 | 32 | 38 | 55 | 29 |
| Gene | Sex | | | | | | | | | | | |
| symbol | Female | Male | Female | Male | Male | Female | Male | Male | Male | Female | Female | Male |
| FASLG | 478.9 | 713.6 | 619.7 | 571.5 | 474.2 | 322.5 | 480.2 | 502.9 | 307.6 | 924.2 | 442.4 | 379.4 |
| CX3CR1 | 24571.5 | 30265.2 | 28708.7 | 23435.1 | 29127.8 | 9004.4 | 22939.5 | 15761.4 | 10347.4 | 23765.0 | 18981.2 | 23972.5 |
| TBX21 | 6753.9 | 10525.3 | 5580.2 | 7759.1 | 6342.7 | 6155.9 | 8214.6 | 7145.2 | 5308.4 | 9864.1 | 5894.7 | 5584.6 |
| ID2 | 2907.5 | 4164.9 | 3465.6 | 2731.4 | 2485.7 | 1596.7 | 1954.9 | 2405.4 | 1663.3 | 4855.6 | 2556.8 | 2791.1 |
| SLAMF7 | 2569.3 | 4244.8 | 4664.8 | 3043.7 | 4237.4 | 1233.0 | 2843.8 | 2370.1 | 1510.5 | 4035.0 | 2688.4 | 2837.0 |
| PRSS23 | 248.9 | 308.4 | 233.0 | 183.8 | 96.6 | 188.9 | 157.3 | 137.8 | 51.8 | 489.3 | 100.9 | 94.4 |
| YWHAQ | 12178.7 | 15459.6 | 11613.8 | 13862.5 | 9757.1 | 8276.2 | 10991.5 | 10985.7 | 9590.5 | 14801.4 | 10540.6 | 9592.9 |
| TARDBP | 9472.5 | 11412.2 | 8978.0 | 12153.6 | 12154.7 | 8069.4 | 10338.5 | 10912.4 | 10339.8 | 10790.8 | 13077.3 | 10771.4 |
| ADRB2 | 1549.3 | 2711.4 | 1459.7 | 1896.1 | 2070.4 | 1245.0 | 1793.6 | 2255.5 | 1093.5 | 3408.0 | 1938.9 | 1310.9 |
| PPP1R8 | 2393.3 | 2370.5 | 2337.2 | 2597.0 | 2563.1 | 1613.1 | 1884.9 | 2213.2 | 1646.9 | 2678.8 | 2436.2 | 2041.2 |
| MMAA | 169.6 | 197.0 | 163.8 | 182.7 | 224.8 | 136.0 | 127.8 | 161.4 | 95.8 | 188.1 | 212.9 | 187.6 |
| SQLE | 375.7 | 302.8 | 271.4 | 678.9 | 532.8 | 363.9 | 550.2 | 666.1 | 547.0 | 543.9 | 525.6 | 488.1 |
| PDHA1 | 3580.4 | 4455.1 | 4025.2 | 3988.0 | 4532.8 | 3562.5 | 4066.9 | 4963.2 | 3761.0 | 4270.5 | 4590.6 | 3797.5 |
| HAVCR2 | 1894.8 | 1673.0 | 1826.9 | 2081.3 | 2072.0 | 1221.0 | 1532.4 | 2068.0 | 1233.2 | 2144.9 | 1244.4 | 2189.8 |
| RACGAP1 | 364.4 | 436.1 | 258.6 | 276.5 | 292.7 | 250.8 | 270.6 | 357.6 | 258.0 | 339.3 | 300.0 | 266.1 |
| AHNAK | 11007.8 | 12380.9 | 12391.6 | 16214.8 | 15383.3 | 7809.8 | 11011.1 | 13799.2 | 10011.8 | 10185.8 | 10937.7 | 10822.5 |
| EDG8 | 252.9 | 121.6 | 265.8 | 397.0 | 179.5 | 183.7 | 240.9 | 231.6 | 97.9 | 252.3 | 231.9 | 218.2 |
| DUSP5 | 169.1 | 163.7 | 162.1 | 223.8 | 386.5 | 131.9 | 214.0 | 211.3 | 153.9 | 254.7 | 147.1 | 131.5 |

TABLE 5

| | Patient | | | | | | | | | | | |
|---|---|---|---|---|---|---|---|---|---|---|---|---|
| | 25 | 26 | 27 | 28 | 29 | 30 | 31 | 32 | 33 | 34 | 35 | 36 |
| | Age | | | | | | | | | | | |
| | 76 | 28 | 44 | 28 | 60 | 41 | 72 | 67 | 55 | 42 | 49 | 36 |
| Gene | Sex | | | | | | | | | | | |
| symbol | Male | Female | Female | Female | Female | Female | Female | Female | Female | Female | Male | Female |
| FASLG | 1090.3 | 557.9 | 320.1 | 434.8 | 404.7 | 426.7 | 586.2 | 257.9 | 423.5 | 321.0 | 246.8 | 262.6 |
| CX3CR1 | 44311.3 | 27862.3 | 24209.0 | 17960.8 | 30156.2 | 24955.7 | 31572.1 | 17272.2 | 23774.0 | 17053.5 | 18408.7 | 8788.8 |
| TBX21 | 11732.1 | 9140.9 | 6046.9 | 5924.7 | 7719.7 | 11525.9 | 10990.6 | 3875.3 | 9148.7 | 4738.1 | 7767.7 | 3709.2 |
| ID2 | 3528.3 | 3770.8 | 2991.7 | 5101.7 | 3144.4 | 3495.3 | 3591.7 | 3035.6 | 3562.5 | 3654.7 | 2579.7 | 2807.2 |
| SLAMF7 | 4876.0 | 3692.2 | 2373.8 | 2844.4 | 2667.3 | 3842.7 | 4628.5 | 1590.0 | 2880.5 | 3572.7 | 2062.9 | 1826.6 |
| PRSS23 | 375.6 | 306.4 | 117.2 | 190.7 | 328.2 | 200.8 | 202.6 | 173.2 | 181.7 | 178.4 | 111.9 | 98.2 |
| YWHAQ | 13194.6 | 10229.2 | 12814.3 | 12947.7 | 13977.3 | 11495.4 | 9786.5 | 11435.8 | 12315.4 | 11018.7 | 7575.4 | 13594.5 |
| TARDBP | 8854.8 | 10597.9 | 11259.0 | 11046.5 | 9661.6 | 10058.5 | 7397.6 | 9948.7 | 11465.3 | 7163.4 | 8502.2 | 10932.3 |
| ADRB2 | 3415.5 | 2500.5 | 2078.5 | 2345.3 | 2716.4 | 2554.2 | 3611.7 | 2243.7 | 2243.6 | 3129.8 | 1865.8 | 1801.3 |
| PPP1R8 | 2367.0 | 2068.8 | 2474.2 | 2353.5 | 2119.6 | 1894.7 | 1885.5 | 2361.4 | 1996.0 | 2286.4 | 1633.5 | 2918.1 |
| MMAA | 191.3 | 149.6 | 129.9 | 162.8 | 161.1 | 165.1 | 198.3 | 144.8 | 137.3 | 157.3 | 136.1 | 153.7 |
| SQLE | 530.2 | 419.7 | 436.4 | 472.1 | 342.7 | 279.1 | 248.8 | 422.6 | 400.6 | 488.1 | 498.1 | 676.4 |
| PDHA1 | 3830.5 | 3535.4 | 3776.2 | 3727.0 | 3425.2 | 4053.0 | 3292.5 | 3536.2 | 3783.7 | 2205.2 | 3460.6 | 3858.5 |
| HAVCR2 | 1766.3 | 1819.1 | 1493.7 | 1633.0 | 1855.1 | 1545.0 | 2258.6 | 2112.3 | 2111.0 | 1677.7 | 1575.1 | 1389.7 |
| RACGAP1 | 371.4 | 310.8 | 345.4 | 403.6 | 288.6 | 284.8 | 252.0 | 303.2 | 345.2 | 446.9 | 277.1 | 431.2 |
| AHNAK | 11749.9 | 8930.3 | 13681.1 | 8265.9 | 11575.0 | 10216.1 | 10001.5 | 11756.2 | 10632.5 | 10592.7 | 10062.1 | 12069.5 |
| EDG8 | 427.2 | 294.4 | 219.6 | 291.2 | 286.8 | 422.9 | 306.1 | 159.2 | 185.2 | 72.4 | 223.0 | 84.9 |
| DUSP5 | 219.3 | 156.1 | 178.0 | 181.2 | 267.5 | 126.7 | 180.6 | 134.6 | 184.7 | 197.5 | 114.4 | 135.1 |

TABLE 6

| | Patient | | | | | | | | | |
|---|---|---|---|---|---|---|---|---|---|---|
| | 37 | 38 | 39 | 40 | 41 | 42 | 43 | 44 | 45 | 46 |
| | Age | | | | | | | | | |
| | 40 | 25 | 35 | 17 | 24 | 64 | 52 | 28 | 60 | 25 |
| Gene | Sex | | | | | | | | | |
| symbol | Female | Male | Female | Female | Male | Female | Male | Female | Female | Female |
| FASLG | 299.0 | 282.2 | 242.6 | 184.5 | 465.6 | 517.6 | 453.7 | 321.1 | 518.3 | 413.9 |
| CX3CR1 | 14718.8 | 18353.6 | 14623.6 | 12982.0 | 27580.7 | 22935.5 | 22416.0 | 12862.0 | 18192.7 | 20173.2 |
| TBX21 | 4106.6 | 7125.4 | 6363.8 | 5038.2 | 6080.1 | 4377.8 | 9514.3 | 3625.8 | 9447.4 | 4678.9 |
| ID2 | 3132.6 | 2207.6 | 2190.2 | 2453.3 | 3114.5 | 3435.4 | 2638.3 | 3242.8 | 3757.5 | 4047.1 |
| SLAMF7 | 2101.3 | 2389.5 | 2253.4 | 2071.6 | 2684.7 | 2820.2 | 2822.6 | 2229.0 | 2933.4 | 2430.1 |
| PRSS23 | 62.3 | 102.7 | 88.8 | 99.1 | 193.4 | 143.3 | 136.2 | 126.2 | 431.8 | 77.9 |
| YWHAQ | 10700.0 | 8652.3 | 7299.2 | 7436.2 | 11773.6 | 14652.8 | 6385.7 | 11040.3 | 10503.2 | 13804.2 |
| TARDBP | 8586.2 | 9219.5 | 8036.5 | 13795.0 | 10453.6 | 11014.1 | 9242.7 | 9214.3 | 8323.0 | 12584.6 |
| ADRB2 | 1867.0 | 2669.2 | 1788.0 | 2016.1 | 1993.4 | 3766.1 | 1682.7 | 1847.4 | 2133.7 | 1890.5 |
| PPP1R8 | 2114.6 | 1703.9 | 1550.3 | 1643.3 | 2446.5 | 2751.1 | 1510.4 | 2375.2 | 1983.7 | 2577.8 |
| MMAA | 143.3 | 89.6 | 125.9 | 120.5 | 192.1 | 210.7 | 146.6 | 173.6 | 213.8 | 218.4 |
| SQLE | 458.2 | 424.0 | 315.8 | 298.6 | 695.0 | 602.3 | 245.7 | 404.8 | 301.6 | 1207.9 |
| PDHA1 | 3239.7 | 3332.1 | 3040.1 | 2787.7 | 4303.5 | 4119.4 | 4271.3 | 3227.3 | 3323.2 | 4559.0 |
| HAVCR2 | 1810.2 | 1379.8 | 1844.6 | 1083.0 | 1898.2 | 2040.6 | 1421.2 | 1666.8 | 2047.5 | 1591.1 |
| RACGAP1 | 278.3 | 217.2 | 289.5 | 237.5 | 368.6 | 433.4 | 222.7 | 376.7 | 337.2 | 407.9 |
| AHNAK | 9528.6 | 9449.1 | 13142.6 | 5758.0 | 12662.2 | 12030.7 | 14017.9 | 7020.4 | 11261.8 | 8581.4 |
| EDG8 | 141.1 | 319.5 | 130.7 | 197.5 | 379.1 | 244.0 | 338.2 | 223.7 | 272.4 | 236.7 |
| DUSP5 | 148.1 | 123.1 | 124.7 | 74.3 | 110.2 | 159.4 | 148.9 | 111.2 | 135.8 | 153.8 |

TABLE 7

| | Healthy individual | | | | | | | | | | |
|---|---|---|---|---|---|---|---|---|---|---|---|
| | 1 | 2 | 3 | 4 | 5 | 6 | 7 | 8 | 9 | 10 | 11 |
| | Age | | | | | | | | | | |
| | 67 | 42 | 23 | 63 | 30 | 67 | 28 | 56 | 26 | 37 | 27 |
| Gene | Sex | | | | | | | | | | |
| symbol | Male | Female | Female | Male | Male | Female | Female | Female | Female | Male | Male |
| FASLG | 802.5 | 911.2 | 628.8 | 769.0 | 586.3 | 877.2 | 657.0 | 722.8 | 861.2 | 614.0 | 902.8 |
| CX3CR1 | 47313.2 | 44733.1 | 30387.4 | 25102.5 | 38277.8 | 29897.2 | 25699.9 | 41247.8 | 33060.1 | 34273.1 | 39255.7 |
| TBX21 | 9569.0 | 20656.8 | 11348.6 | 7203.9 | 4492.0 | 12474.1 | 5971.4 | 7267.3 | 11429.1 | 6892.5 | 14548.6 |
| ID2 | 3894.1 | 8887.1 | 3948.3 | 4326.9 | 4728.6 | 5466.8 | 2830.1 | 5309.8 | 5768.3 | 4050.9 | 4856.0 |
| SLAMF7 | 3829.1 | 5102.4 | 3853.1 | 4005.8 | 3628.1 | 4455.8 | 7354.6 | 5267.0 | 4074.2 | 3641.9 | 4814.6 |
| PRSS23 | 485.4 | 1090.9 | 235.4 | 257.4 | 248.4 | 642.4 | 186.4 | 534.0 | 518.3 | 312.0 | 611.5 |
| YWHAQ | 13960.4 | 19673.5 | 12143.1 | 17904.8 | 14750.6 | 17439.6 | 14597.9 | 13814.8 | 18710.8 | 14643.8 | 13562.3 |
| TARDBP | 12892.6 | 13249.0 | 13528.2 | 11860.5 | 10101.4 | 10518.1 | 8907.8 | 11469.2 | 13105.9 | 9300.2 | 15851.7 |
| ADRB2 | 3760.0 | 4731.9 | 2657.6 | 2290.2 | 1827.2 | 3789.3 | 3071.0 | 1919.5 | 3015.7 | 1588.7 | 3931.5 |
| PPP1R8 | 3135.2 | 2253.9 | 2422.0 | 3145.7 | 2517.4 | 2930.8 | 2384.1 | 2933.2 | 3344.8 | 2612.0 | 3288.0 |
| MMAA | 232.8 | 181.7 | 232.1 | 266.7 | 218.8 | 215.3 | 300.9 | 201.3 | 255.0 | 203.9 | 235.0 |
| SQLE | 867.4 | 590.6 | 611.6 | 883.7 | 425.8 | 718.1 | 854.3 | 475.8 | 674.4 | 434.3 | 854.1 |
| PDHA1 | 4601.6 | 4736.6 | 4895.8 | 4724.8 | 4534.8 | 4208.3 | 4017.6 | 4295.5 | 4863.3 | 4050.9 | 4756.1 |
| HAVCR2 | 2707.7 | 3044.1 | 1924.7 | 2063.3 | 2308.5 | 1556.2 | 3440.1 | 2016.3 | 2768.8 | 1928.2 | 2359.4 |
| RACGAP1 | 359.7 | 370.7 | 333.1 | 441.3 | 284.8 | 400.8 | 313.9 | 315.5 | 382.0 | 319.0 | 485.1 |
| AHNAK | 17431.1 | 11142.6 | 10929.9 | 16932.7 | 9523.5 | 15994.3 | 8241.3 | 14073.1 | 13499.3 | 13536.4 | 14928.6 |
| EDG8 | 388.7 | 374.1 | 416.6 | 315.8 | 154.0 | 587.6 | 290.0 | 336.8 | 682.7 | 289.4 | 662.4 |
| DUSP5 | 304.3 | 163.4 | 203.3 | 191.7 | 92.1 | 176.9 | 1222.2 | 188.7 | 174.0 | 161.0 | 194.3 |

TABLE 8

| | Healthy individual | | | | | | | | | | |
|---|---|---|---|---|---|---|---|---|---|---|---|
| | 12 | 13 | 14 | 15 | 16 | 17 | 18 | 19 | 20 | 21 | 22 |
| | Age | | | | | | | | | | |
| | 53 | 57 | 36 | 33 | 44 | 45 | 38 | 38 | 36 | 56 | 23 |
| Gene | Sex | | | | | | | | | | |
| symbol | Female | Female | Male | Male | Female | Female | Male | Male | Female | Male | Female |
| FASLG | 527.8 | 1438.4 | 1004.7 | 675.5 | 481.7 | 1282.1 | 694.9 | 604.2 | 599.8 | 1046.4 | 1536.4 |
| CX3CR1 | 26966.3 | 40711.8 | 37130.5 | 35742.2 | 19826.4 | 64658.3 | 32751.9 | 36223.8 | 32633.4 | 46100.1 | 51701.9 |
| TBX21 | 6581.4 | 14273.3 | 10786.3 | 10691.0 | 5744.5 | 12283.4 | 7185.6 | 8358.7 | 8547.3 | 11550.5 | 16172.6 |
| ID2 | 3704.9 | 5700.7 | 4456.3 | 5081.0 | 4993.3 | 4700.2 | 4289.9 | 3111.0 | 3673.7 | 3706.4 | 6050.4 |
| SLAMF7 | 3574.1 | 6310.4 | 4480.7 | 4667.9 | 3365.5 | 9082.4 | 3325.7 | 2860.2 | 3797.0 | 5390.2 | 5354.1 |
| PRSS23 | 215.8 | 690.9 | 397.9 | 441.1 | 355.2 | 416.4 | 255.6 | 260.1 | 297.6 | 612.4 | 768.8 |
| YWHAQ | 12294.1 | 15630.0 | 13934.2 | 14607.8 | 12105.0 | 18752.2 | 15232.0 | 17105.7 | 18354.3 | 16503.5 | 17289.0 |
| TARDBP | 10319.9 | 11831.3 | 11638.0 | 10071.7 | 9990.2 | 13491.4 | 14209.1 | 15789.1 | 14009.2 | 11109.8 | 11327.6 |
| ADRB2 | 1521.3 | 4011.7 | 3410.6 | 2383.7 | 1596.1 | 2646.6 | 2930.7 | 2742.3 | 3032.2 | 3698.0 | 4373.2 |
| PPP1R8 | 2545.0 | 3440.5 | 3162.8 | 2467.4 | 2924.6 | 2973.4 | 3004.6 | 2138.8 | 2387.4 | 2932.7 | 2949.1 |
| MMAA | 194.7 | 286.1 | 192.4 | 198.2 | 191.8 | 267.3 | 215.8 | 167.2 | 192.3 | 221.5 | 264.8 |
| SQLE | 446.3 | 847.3 | 473.6 | 530.1 | 586.0 | 574.7 | 710.8 | 703.4 | 531.0 | 972.5 | 651.3 |
| PDHA1 | 3710.7 | 4823.6 | 4326.5 | 3931.1 | 3569.7 | 5461.0 | 5045.7 | 5060.3 | 5041.8 | 4394.4 | 4063.2 |
| HAVCR2 | 1829.4 | 2812.6 | 2447.7 | 2118.9 | 1549.2 | 3738.7 | 2388.9 | 1690.4 | 1324.5 | 2744.3 | 2299.5 |
| RACGAP1 | 398.5 | 443.4 | 364.5 | 434.4 | 361.5 | 640.5 | 363.3 | 349.5 | 401.7 | 435.0 | 358.2 |
| AHNAK | 12857.6 | 18017.5 | 13931.4 | 11858.1 | 14205.8 | 11870.3 | 15944.5 | 14145.3 | 13079.8 | 16970.8 | 8325.3 |
| EDG8 | 186.2 | 671.0 | 538.3 | 482.5 | 169.4 | 333.9 | 240.4 | 213.8 | 216.5 | 540.6 | 777.3 |
| DUSP5 | 182.7 | 259.1 | 152.7 | 223.5 | 159.4 | 325.4 | 143.2 | 133.7 | 229.1 | 253.9 | 171.5 |

TABLE 9

| | Healthy individual | | | | | | | | | | |
|---|---|---|---|---|---|---|---|---|---|---|---|
| | 23 | 24 | 25 | 26 | 27 | 28 | 29 | 30 | 31 | 32 | 33 |
| | Age | | | | | | | | | | |
| | 23 | 23 | 23 | 23 | 22 | 21 | 21 | 21 | 21 | 21 | 24 |
| Gene | Sex | | | | | | | | | | |
| symbol | Female | Female | Female | Female | Female | Female | Female | Female | Female | Female | Male |
| FASLG | 691.8 | 653.4 | 937.5 | 791.8 | 879.7 | 1552.9 | 494.3 | 584.0 | 721.6 | 570.9 | 1007.2 |
| CX3CR1 | 27378.1 | 44508.9 | 36013.5 | 22814.5 | 39713.1 | 45067.5 | 30031.9 | 26967.6 | 23230.3 | 29833.3 | 44211.4 |
| TBX21 | 8227.7 | 11864.7 | 11186.0 | 8858.8 | 12372.1 | 26473.8 | 9806.2 | 10004.1 | 9310.1 | 10361.2 | 12064.6 |
| ID2 | 5737.5 | 5333.6 | 6128.5 | 5503.8 | 4444.5 | 8367.2 | 4444.0 | 6854.9 | 4089.6 | 3970.6 | 4419.0 |
| SLAMF7 | 3833.3 | 4324.5 | 4643.7 | 4292.0 | 3916.3 | 6273.0 | 3452.0 | 4050.5 | 3973.4 | 4284.1 | 4592.5 |
| PRSS23 | 210.8 | 187.5 | 423.0 | 226.5 | 456.6 | 634.0 | 193.9 | 290.3 | 284.5 | 274.3 | 417.3 |
| YWHAQ | 16963.0 | 15879.7 | 13178.3 | 15882.7 | 11664.5 | 17053.5 | 14836.2 | 16453.4 | 14452.9 | 13083.7 | 15189.0 |
| TARDBP | 13013.8 | 18059.2 | 12279.5 | 15469.0 | 13521.4 | 14579.2 | 15257.9 | 13487.0 | 13395.8 | 15102.3 | 10156.6 |
| ADRB2 | 4617.5 | 3579.5 | 3955.1 | 3429.3 | 3286.9 | 4805.3 | 3280.1 | 3328.8 | 3860.4 | 3347.4 | 4848.3 |
| PPP1R8 | 3047.7 | 2496.0 | 2954.3 | 2939.5 | 2357.6 | 2408.1 | 2382.1 | 2944.1 | 2605.9 | 2327.8 | 2807.6 |
| MMAA | 279.7 | 276.5 | 259.8 | 287.5 | 204.0 | 201.2 | 167.7 | 270.0 | 229.3 | 145.0 | 229.3 |
| SQLE | 902.7 | 928.1 | 605.9 | 873.2 | 492.9 | 770.1 | 846.2 | 645.3 | 747.2 | 677.4 | 624.4 |
| PDHA1 | 5298.9 | 6023.5 | 4992.7 | 5749.9 | 4349.6 | 4652.2 | 5133.8 | 5233.8 | 4958.6 | 4609.1 | 4630.6 |
| HAVCR2 | 2668.1 | 3430.7 | 1766.1 | 2193.9 | 1616.6 | 2347.6 | 1928.7 | 1765.9 | 2131.8 | 2030.0 | 2531.0 |
| RACGAP1 | 377.4 | 532.9 | 360.5 | 423.0 | 334.3 | 447.1 | 420.5 | 377.0 | 422.7 | 454.6 | 421.7 |
| AHNAK | 13382.7 | 8695.2 | 9243.7 | 10701.1 | 9290.9 | 11408.0 | 12525.2 | 10100.7 | 16175.3 | 15979.7 | 13382.4 |
| EDG8 | 160.4 | 265.1 | 304.5 | 161.8 | 450.9 | 494.1 | 153.1 | 216.0 | 238.4 | 180.5 | 487.8 |
| DUSP5 | 175.8 | 251.3 | 225.6 | 194.6 | 190.2 | 180.6 | 193.7 | 234.0 | 250.1 | 259.1 | 219.0 |

TABLE 10

| | Healthy individual | | | | | | | | | | |
|---|---|---|---|---|---|---|---|---|---|---|---|
| | 34 | 35 | 36 | 37 | 38 | 39 | 40 | 41 | 42 | 43 | 44 |
| | Age | | | | | | | | | | |
| | 24 | 24 | 23 | 23 | 23 | 23 | 23 | 23 | 23 | 22 | 22 |
| Gene | Sex | | | | | | | | | | |
| symbol | Male | Male | Male | Male | Male | Male | Male | Male | Male | Male | Male |
| FASLG | 644.8 | 1776.5 | 999.7 | 650.3 | 1634.9 | 775.6 | 747.5 | 496.8 | 790.8 | 918.4 | 703.9 |
| CX3CR1 | 30925.5 | 46315.2 | 38138.0 | 24300.7 | 50661.6 | 29370.2 | 27325.7 | 27612.4 | 26246.5 | 37049.4 | 43961.4 |
| TBX21 | 7768.6 | 23905.4 | 9762.4 | 6729.0 | 19924.7 | 8187.5 | 9950.3 | 9160.2 | 8643.5 | 15377.7 | 8357.8 |
| ID2 | 3817.7 | 9065.9 | 4567.9 | 4233.6 | 5747.1 | 4035.6 | 3392.2 | 3892.9 | 3661.2 | 6044.8 | 4307.9 |
| SLAMF7 | 7392.0 | 7381.6 | 3754.8 | 3009.9 | 4975.5 | 4284.5 | 3974.5 | 3217.1 | 3680.1 | 5699.8 | 6448.3 |
| PRSS23 | 232.6 | 827.5 | 345.8 | 336.6 | 768.2 | 444.2 | 223.4 | 147.0 | 329.3 | 449.4 | 134.1 |
| YWHAQ | 14357.9 | 15762.3 | 13956.0 | 12794.1 | 15542.7 | 14595.7 | 13336.7 | 13802.1 | 16022.9 | 17289.5 | 14483.3 |
| TARDBP | 12697.6 | 16145.1 | 10746.4 | 12749.0 | 12945.1 | 10872.2 | 11451.6 | 13819.2 | 12035.1 | 13148.1 | 11656.2 |
| ADRB2 | 3364.0 | 5984.9 | 3399.4 | 3770.1 | 3891.6 | 3787.4 | 3828.3 | 3411.4 | 4186.0 | 3637.9 | 4340.1 |
| PPP1R8 | 2465.9 | 3518.3 | 2564.6 | 2638.0 | 2763.3 | 3064.5 | 2763.9 | 2893.8 | 2945.1 | 3126.0 | 2965.3 |
| MMAA | 286.9 | 253.6 | 210.5 | 210.5 | 219.9 | 211.2 | 190.3 | 197.6 | 223.1 | 203.3 | 258.2 |
| SQLE | 796.3 | 871.0 | 700.8 | 900.2 | 1032.6 | 547.1 | 682.0 | 718.5 | 593.6 | 788.5 | 1055.7 |
| PDHA1 | 4915.3 | 5274.3 | 4065.5 | 4470.7 | 4383.4 | 4045.5 | 4289.6 | 5136.8 | 4049.5 | 4965.9 | 4822.5 |
| HAVCR2 | 2397.1 | 2793.4 | 1752.3 | 2253.6 | 2339.2 | 1493.7 | 1992.2 | 1814.5 | 1460.5 | 1916.4 | 3452.5 |
| RACGAP1 | 391.2 | 555.5 | 345.6 | 522.4 | 418.2 | 410.6 | 409.2 | 450.2 | 392.9 | 550.4 | 370.5 |
| AHNAK | 9759.3 | 17921.7 | 10923.2 | 13290.5 | 9296.2 | 10647.3 | 11560.4 | 15309.3 | 11614.3 | 13948.0 | 11311.8 |
| EDG8 | 292.7 | 722.1 | 369.8 | 166.4 | 921.6 | 388.8 | 421.5 | 345.3 | 420.6 | 498.7 | 330.5 |
| DUSP5 | 276.0 | 212.4 | 272.6 | 142.0 | 285.0 | 164.1 | 236.3 | 143.7 | 177.2 | 293.8 | 359.7 |

TABLE 11

| | Healthy individual | | | | | | | | | | |
|---|---|---|---|---|---|---|---|---|---|---|---|
| | 45 | 46 | 47 | 48 | 49 | 50 | 51 | 52 | 53 | 54 | 55 |
| | Age | | | | | | | | | | |
| | 22 | 22 | 21 | 71 | 32 | 49 | 64 | 26 | 30 | 57 | 26 |
| Gene | Sex | | | | | | | | | | |
| symbol | Male | Male | Male | Female | Female | Female | Male | Female | Female | Female | Female |
| FASLG | 1069.0 | 2092.5 | 717.6 | 793.7 | 776.6 | 619.9 | 1161.3 | 607.6 | 937.5 | 787.6 | 1241.0 |
| CX3CR1 | 38115.4 | 17277.6 | 27255.3 | 33468.8 | 40772.1 | 28043.1 | 45510.7 | 35024.2 | 35327.6 | 35376.2 | 54888.5 |
| TBX21 | 10967.6 | 4968.9 | 10608.1 | 8910.2 | 14990.9 | 7243.4 | 19120.0 | 11706.3 | 14972.1 | 15660.6 | 23043.3 |
| ID2 | 4940.3 | 4908.5 | 5084.1 | 3912.9 | 6857.4 | 3698.9 | 5552.2 | 3709.1 | 7595.6 | 4922.9 | 8452.9 |
| SLAMF7 | 4394.7 | 1606.9 | 3450.3 | 3765.1 | 5096.6 | 3666.8 | 5053.7 | 4248.4 | 4391.6 | 4475.3 | 5217.8 |
| PRSS23 | 439.9 | 166.8 | 260.4 | 330.5 | 592.1 | 257.5 | 658.7 | 347.2 | 557.3 | 991.0 | 936.9 |
| YWHAQ | 14416.2 | 12376.2 | 12853.6 | 16050.5 | 16342.2 | 15585.2 | 14853.7 | 13809.0 | 14541.3 | 16287.1 | 17209.4 |
| TARDBP | 11203.8 | 11894.5 | 12594.2 | 12216.4 | 17341.5 | 11944.5 | 12863.9 | 12590.4 | 13193.5 | 13054.3 | 16336.8 |
| ADRB2 | 4564.2 | 3250.1 | 2684.5 | 2969.9 | 3402.8 | 3168.4 | 3628.2 | 2710.1 | 3090.3 | 3557.3 | 6293.4 |
| PPP1R8 | 3169.9 | 3256.3 | 3131.3 | 2784.0 | 2693.2 | 3051.8 | 2513.0 | 2329.7 | 2770.3 | 2927.2 | 2781.1 |
| MMAA | 215.7 | 204.1 | 189.8 | 230.9 | 190.0 | 227.6 | 226.5 | 216.0 | 187.6 | 219.3 | 254.5 |
| SQLE | 520.9 | 771.2 | 577.8 | 518.0 | 811.5 | 839.5 | 478.8 | 519.3 | 755.6 | 685.6 | 779.1 |
| PDHA1 | 4266.9 | 4320.2 | 4764.6 | 4781.7 | 5253.9 | 4283.8 | 4453.7 | 4017.3 | 4696.1 | 4266.5 | 5461.2 |
| HAVCR2 | 2416.7 | 1546.4 | 2189.0 | 2147.2 | 2258.8 | 1194.6 | 2473.8 | 1673.6 | 3145.7 | 1673.6 | 3104.0 |
| RACGAP1 | 378.1 | 364.6 | 405.1 | 390.4 | 516.4 | 377.2 | 398.7 | 335.1 | 345.5 | 459.4 | 526.4 |
| AHNAK | 10921.5 | 9396.4 | 10311.2 | 13374.0 | 13578.6 | 12313.5 | 14181.1 | 12543.2 | 8838.1 | 14099.6 | 14883.8 |
| EDG8 | 519.6 | 202.7 | 521.6 | 263.2 | 122.5 | 297.0 | 472.9 | 484.9 | 327.8 | 232.8 | 353.1 |
| DUSP5 | 290.2 | 112.2 | 148.6 | 194.8 | 293.6 | 231.4 | 171.4 | 240.8 | 145.3 | 190.7 | 217.5 |

TABLE 12

| | Healthy individual | | | | | | | | | | |
|---|---|---|---|---|---|---|---|---|---|---|---|
| | 56 | 57 | 58 | 59 | 60 | 61 | 62 | 63 | 64 | 65 | 66 |
| | Age | | | | | | | | | | |
| | 33 | 43 | 44 | 55 | 33 | 41 | 45 | 44 | 46 | 41 | 65 |
| Gene | Sex | | | | | | | | | | |
| symbol | Female | Female | Female | Female | Male | Female | Male | Male | Male | Male | Female |
| FASLG | 564.9 | 767.0 | 601.9 | 456.9 | 1154.9 | 945.0 | 877.4 | 531.1 | 646.9 | 935.6 | 865.3 |
| CX3CR1 | 14540.0 | 30045.5 | 27067.0 | 34391.8 | 46650.8 | 44637.2 | 32216.7 | 36687.7 | 35873.6 | 27843.2 | 46312.8 |
| TBX21 | 10001.4 | 13792.0 | 12929.7 | 13360.6 | 24421.8 | 22327.6 | 19614.1 | 10920.8 | 13315.0 | 11563.9 | 11061.2 |
| ID2 | 5641.6 | 4223.4 | 5496.3 | 2777.4 | 9892.5 | 6573.9 | 6940.2 | 3737.8 | 5081.4 | 3845.8 | 6762.5 |
| SLAMF7 | 3766.9 | 4929.0 | 4205.2 | 4155.8 | 6926.9 | 5318.2 | 6281.3 | 3325.3 | 3384.1 | 3071.3 | 6192.2 |
| PRSS23 | 299.9 | 286.6 | 521.9 | 137.1 | 811.8 | 844.6 | 655.0 | 511.3 | 369.4 | 354.7 | 533.1 |
| YWHAQ | 13401.7 | 15616.5 | 18340.3 | 9054.9 | 20724.2 | 17176.2 | 15094.4 | 15132.5 | 11365.7 | 12986.7 | 18753.5 |
| TARDBP | 14273.7 | 14366.2 | 14076.1 | 11892.1 | 14207.6 | 15346.3 | 15635.9 | 14088.1 | 12660.6 | 13476.2 | 10532.0 |
| ADRB2 | 2496.3 | 3305.6 | 3105.2 | 2766.5 | 7088.9 | 4591.3 | 4520.7 | 4107.4 | 2307.1 | 2829.2 | 3209.9 |
| PPP1R8 | 3287.7 | 2387.8 | 2732.7 | 1593.3 | 3006.3 | 3133.2 | 3374.2 | 2271.2 | 2617.7 | 2329.3 | 3039.9 |
| MMAA | 164.9 | 139.2 | 212.6 | 141.5 | 231.2 | 196.5 | 180.5 | 188.8 | 145.0 | 253.0 | 224.0 |
| SQLE | 828.3 | 587.7 | 610.3 | 468.8 | 766.9 | 915.1 | 774.4 | 780.2 | 612.4 | 537.5 | 523.8 |
| PDHA1 | 4933.3 | 4417.5 | 4486.7 | 4582.2 | 4847.8 | 6634.9 | 6032.5 | 5427.0 | 4655.2 | 4579.0 | 4796.3 |
| HAVCR2 | 2319.8 | 1660.2 | 2071.0 | 1332.6 | 1914.3 | 2234.7 | 2569.1 | 1500.1 | 2849.8 | 1583.8 | 1949.1 |
| RACGAP1 | 521.9 | 408.9 | 418.3 | 342.7 | 536.3 | 927.6 | 698.7 | 367.8 | 439.6 | 424.6 | 580.3 |
| AHNAK | 15079.6 | 14892.0 | 9608.4 | 17838.8 | 19443.0 | 17046.6 | 20052.4 | 17744.8 | 17033.5 | 17513.2 | 16510.1 |
| EDG8 | 171.3 | 241.9 | 147.6 | 397.1 | 435.2 | 340.4 | 359.8 | 248.3 | 416.7 | 399.6 | 478.8 |
| DUSP5 | 297.8 | 255.8 | 266.9 | 339.8 | 272.8 | 248.2 | 344.9 | 140.9 | 176.4 | 270.8 | 363.1 |

TABLE 13

| | Healthy individual | | | | | | | | | | |
|---|---|---|---|---|---|---|---|---|---|---|---|
| | 67 | 68 | 69 | 70 | 71 | 72 | 73 | 74 | 75 | 76 | 77 |
| | Age | | | | | | | | | | |
| | 42 | 41 | 43 | 40 | 45 | 65 | 72 | 65 | 39 | 41 | 41 |
| Gene | Sex | | | | | | | | | | |
| symbol | Female | Female | Female | Male | Male | Male | Male | Male | Female | Male | Male |
| FASLG | 858.0 | 485.9 | 807.2 | 422.6 | 603.0 | 799.7 | 588.0 | 873.1 | 916.4 | 818.8 | 858.5 |
| CX3CR1 | 31490.7 | 21889.5 | 36128.0 | 30478.7 | 31085.0 | 34837.4 | 23537.0 | 41548.0 | 30524.6 | 26719.4 | 43429.7 |
| TBX21 | 13747.6 | 7277.6 | 12547.4 | 9990.0 | 9514.9 | 13462.4 | 7845.4 | 13990.7 | 10751.5 | 11087.8 | 14672.1 |
| ID2 | 3918.9 | 3283.0 | 4577.6 | 3413.4 | 3408.5 | 5133.5 | 3734.7 | 4762.6 | 5210.6 | 5450.0 | 4089.2 |
| SLAMF7 | 4122.5 | 3101.1 | 4882.3 | 3561.4 | 4429.0 | 4344.2 | 3366.2 | 5091.2 | 4083.8 | 4150.1 | 6228.5 |
| PRSS23 | 302.9 | 173.2 | 681.2 | 175.8 | 242.9 | 442.1 | 247.2 | 387.7 | 468.7 | 298.5 | 344.3 |
| YWHAQ | 13297.4 | 11983.3 | 15927.5 | 11546.3 | 14928.4 | 13817.9 | 13651.1 | 12335.9 | 15925.5 | 16261.8 | 14983.9 |
| TARDBP | 13357.4 | 12089.5 | 11489.3 | 10403.8 | 9797.5 | 11313.9 | 9276.6 | 11378.6 | 10922.8 | 16022.7 | 12674.5 |
| ADRB2 | 3855.3 | 2248.8 | 2747.3 | 1720.4 | 2173.3 | 3234.8 | 1555.6 | 2797.7 | 3041.8 | 4073.4 | 3577.8 |
| PPP1R8 | 2756.1 | 2457.1 | 2417.5 | 2235.5 | 2445.3 | 3019.5 | 2287.6 | 2622.9 | 2464.1 | 3756.4 | 2685.6 |
| MMAA | 200.4 | 204.5 | 195.2 | 186.2 | 200.9 | 236.9 | 215.1 | 230.1 | 241.4 | 246.6 | 216.5 |
| SQLE | 609.1 | 493.1 | 393.0 | 511.0 | 286.0 | 778.7 | 368.6 | 585.7 | 445.6 | 870.6 | 526.1 |
| PDHA1 | 4898.6 | 4566.6 | 4500.8 | 4072.7 | 3717.6 | 4698.9 | 3609.6 | 4510.3 | 3598.6 | 5690.8 | 4732.9 |
| HAVCR2 | 2290.2 | 2150.0 | 1680.1 | 2105.9 | 1688.7 | 3040.7 | 2137.3 | 2507.0 | 2458.1 | 1776.9 | 1829.6 |
| RACGAP1 | 324.7 | 363.8 | 407.7 | 340.2 | 296.9 | 392.6 | 297.4 | 351.0 | 352.7 | 604.7 | 511.7 |
| AHNAK | 17229.9 | 12846.2 | 12202.5 | 17430.8 | 10900.1 | 14302.4 | 11268.8 | 16905.5 | 8658.5 | 19394.5 | 17766.9 |
| EDG8 | 500.0 | 278.9 | 439.9 | 351.0 | 336.5 | 469.5 | 388.2 | 647.9 | 553.4 | 227.5 | 804.7 |
| DUSP5 | 191.2 | 231.8 | 221.4 | 170.9 | 178.6 | 335.2 | 167.9 | 266.6 | 232.3 | 219.8 | 208.0 |

TABLE 14

| | Healthy individual | | | | | | | | | | |
|---|---|---|---|---|---|---|---|---|---|---|---|
| | 78 | 79 | 80 | 81 | 82 | 83 | 84 | 85 | 86 | 87 | 88 |
| | Age | | | | | | | | | | |
| | 40 | 46 | 42 | 46 | 44 | 43 | 45 | 41 | 49 | 40 | 88 |
| Gene | Sex | | | | | | | | | | |
| symbol | Male | Male | Male | Female | Female | Male | Male | Male | Male | Male | Female |
| FASLG | 875.1 | 401.7 | 779.1 | 1411.8 | 721.5 | 664.5 | 850.6 | 648.1 | 403.5 | 402.8 | 2733.2 |
| CX3CR1 | 56593.0 | 23588.3 | 34817.0 | 56971.7 | 37923.7 | 24018.1 | 34917.7 | 33606.3 | 31497.4 | 23517.8 | 67456.7 |
| TBX21 | 17078.4 | 5906.3 | 9320.4 | 24210.5 | 13041.1 | 10521.8 | 13345.4 | 13094.6 | 8519.5 | 10676.3 | 26494.1 |
| ID2 | 3758.6 | 2858.4 | 4105.4 | 6498.1 | 5047.5 | 4093.0 | 5645.2 | 4782.7 | 3091.5 | 3870.9 | 10853.5 |
| SLAMF7 | 5423.9 | 2378.2 | 4186.0 | 6139.0 | 5003.8 | 4137.3 | 4813.7 | 5241.1 | 2767.2 | 3164.3 | 12570.2 |
| PRSS23 | 428.5 | 91.9 | 304.4 | 916.3 | 362.6 | 239.1 | 383.1 | 240.9 | 165.7 | 92.0 | 1704.0 |
| YWHAQ | 14294.5 | 11984.1 | 12535.8 | 17486.9 | 14408.9 | 15515.9 | 14850.4 | 16221.7 | 11086.1 | 12857.8 | 25731.8 |
| TARDBP | 12544.9 | 13589.1 | 12556.7 | 12978.8 | 11775.2 | 13422.2 | 12281.4 | 14217.7 | 11111.2 | 11464.6 | 13368.1 |
| ADRB2 | 3413.9 | 2080.9 | 3377.0 | 6070.9 | 3447.0 | 2783.2 | 2624.2 | 2620.6 | 2270.4 | 2196.2 | 8279.2 |
| PPP1R8 | 2137.1 | 2658.1 | 2802.9 | 2646.8 | 2770.0 | 2977.3 | 2704.0 | 2663.1 | 2132.8 | 2327.2 | 4000.4 |
| MMAA | 207.1 | 175.5 | 216.8 | 238.9 | 236.6 | 228.1 | 191.8 | 248.6 | 174.9 | 180.4 | 307.4 |
| SQLE | 507.8 | 607.1 | 610.6 | 700.0 | 478.6 | 1028.1 | 503.2 | 762.2 | 634.6 | 598.7 | 926.1 |
| PDHA1 | 4607.6 | 4418.7 | 4734.3 | 5113.9 | 4436.0 | 6112.6 | 4411.5 | 5586.8 | 4474.7 | 4397.9 | 5770.9 |
| HAVCR2 | 3515.6 | 1387.7 | 2021.6 | 3079.3 | 2700.7 | 2634.8 | 2456.9 | 1796.5 | 1759.1 | 2075.4 | 6452.7 |
| RACGAP1 | 441.3 | 450.2 | 413.3 | 451.3 | 334.4 | 636.8 | 397.5 | 485.8 | 334.4 | 341.1 | 504.6 |
| AHNAK | 13475.9 | 12719.0 | 13168.6 | 18218.6 | 10510.0 | 16373.4 | 14414.5 | 18205.7 | 11060.3 | 12554.2 | 18399.6 |
| EDG8 | 681.7 | 347.1 | 485.5 | 1065.1 | 498.1 | 382.1 | 461.7 | 505.9 | 321.1 | 354.0 | 1443.4 |
| DUSP5 | 345.3 | 211.3 | 298.3 | 393.7 | 358.1 | 307.9 | 267.4 | 294.2 | 228.8 | 217.4 | 482.6 |

TABLE 15

| | Healthy individual | | | | | | | | | | |
|---|---|---|---|---|---|---|---|---|---|---|---|
| | 89 | 90 | 91 | 92 | 93 | 94 | 95 | 96 | 97 | 98 | 99 |
| | Age | | | | | | | | | | |
| | 59 | 71 | 70 | 74 | 73 | 64 | 63 | 54 | 76 | 73 | 70 |
| Gene | Sex | | | | | | | | | | |
| symbol | Female | Female | Female | Female | Female | Female | Female | Female | Female | Female | Female |
| FASLG | 1566.2 | 1813.2 | 1157.2 | 1327.6 | 1240.2 | 1504.1 | 652.5 | 573.4 | 1788.9 | 701.7 | 1106.3 |
| CX3CR1 | 54211.4 | 51509.2 | 38444.7 | 48907.0 | 48816.6 | 51931.6 | 23636.1 | 24787.0 | 54545.4 | 32241.2 | 54616.3 |
| TBX21 | 18081.4 | 18024.4 | 14576.9 | 13056.3 | 10327.7 | 16246.6 | 6209.3 | 6080.4 | 19183.7 | 6439.0 | 13176.0 |
| ID2 | 8009.4 | 5228.9 | 6369.5 | 5586.8 | 4375.9 | 7653.6 | 3613.4 | 4032.1 | 5982.1 | 3950.4 | 5573.6 |
| SLAMF7 | 8230.0 | 7138.5 | 4927.7 | 6705.3 | 4954.3 | 6085.2 | 3219.4 | 3600.1 | 8246.2 | 4325.7 | 6821.1 |
| PRSS23 | 998.2 | 1217.1 | 858.3 | 525.6 | 614.8 | 944.0 | 398.8 | 277.8 | 1049.0 | 373.3 | 1219.4 |
| YWHAQ | 20088.4 | 17007.8 | 21412.8 | 14852.5 | 20463.7 | 23272.3 | 13098.4 | 17315.4 | 18841.7 | 15750.4 | 23240.9 |
| TARDBP | 13389.8 | 14660.7 | 12101.1 | 9013.1 | 13520.1 | 12732.5 | 12807.5 | 13040.5 | 12273.4 | 11636.4 | 14286.4 |
| ADRB2 | 4009.8 | 5070.8 | 4144.9 | 3303.9 | 3303.4 | 5355.3 | 2604.5 | 2133.2 | 5136.3 | 2151.6 | 4098.5 |
| PPP1R8 | 3707.7 | 3440.6 | 3346.1 | 2686.7 | 2958.7 | 3714.4 | 3050.6 | 3448.4 | 2835.3 | 2836.4 | 3406.5 |
| MMAA | 262.9 | 276.2 | 277.1 | 209.5 | 283.2 | 262.4 | 241.2 | 231.5 | 251.0 | 262.4 | 303.7 |
| SQLE | 1056.9 | 860.9 | 733.3 | 449.5 | 717.8 | 896.0 | 1087.4 | 647.8 | 695.9 | 651.4 | 1002.7 |
| PDHA1 | 5339.0 | 5121.2 | 5135.5 | 3997.1 | 4226.0 | 4997.9 | 4460.2 | 4081.4 | 4334.7 | 3668.4 | 4910.4 |
| HAVCR2 | 2209.7 | 3012.2 | 3199.0 | 3458.3 | 1946.9 | 1540.5 | 1378.9 | 1534.9 | 2248.6 | 2046.9 | 4098.3 |
| RACGAP1 | 530.1 | 407.7 | 547.1 | 377.8 | 424.7 | 555.8 | 441.6 | 388.6 | 469.7 | 409.9 | 504.5 |
| AHNAK | 17589.2 | 14657.2 | 19020.2 | 14259.0 | 14850.9 | 24588.9 | 10896.5 | 14021.2 | 15749.1 | 12479.9 | 19612.0 |
| EDG8 | 862.7 | 791.6 | 724.2 | 688.6 | 513.7 | 688.9 | 436.8 | 256.5 | 1085.9 | 443.0 | 733.7 |
| DUSP5 | 227.2 | 256.2 | 449.4 | 197.7 | 230.0 | 272.9 | 185.6 | 235.0 | 368.5 | 256.2 | 323.1 |

TABLE 16

| | Healthy individual | | | | | | | | | | |
|---|---|---|---|---|---|---|---|---|---|---|---|
| | 100 | 101 | 102 | 103 | 104 | 105 | 106 | 107 | 108 | 109 | 110 |
| | Age | | | | | | | | | | |
| | 74 | 66 | 63 | 75 | 60 | 76 | 73 | 60 | 65 | 61 | 65 |
| Gene | Sex | | | | | | | | | | |
| symbol | Female | Female | Female | Female | Female | Female | Female | Female | Female | Female | Female |
| FASLG | 886.3 | 514.6 | 1779.8 | 1100.2 | 964.3 | 1609.0 | 1624.6 | 657.1 | 960.1 | 669.9 | 1510.6 |
| CX3CR1 | 41835.5 | 28271.5 | 60336.2 | 35450.3 | 34264.0 | 60512.2 | 52179.8 | 28672.9 | 38786.8 | 33015.0 | 53172.3 |
| TBX21 | 10448.9 | 5259.3 | 20570.8 | 8930.7 | 7857.2 | 21966.3 | 22771.3 | 7657.6 | 9287.6 | 8090.2 | 16665.2 |
| ID2 | 4560.7 | 4221.1 | 7798.7 | 6861.7 | 6773.6 | 7970.2 | 10024.7 | 5456.4 | 4599.1 | 3308.9 | 6875.3 |
| SLAMF7 | 5223.2 | 3240.5 | 9608.5 | 5761.4 | 6363.1 | 7044.2 | 7540.7 | 3702.0 | 4478.0 | 3355.8 | 6624.5 |
| PRSS23 | 565.8 | 213.1 | 1085.1 | 401.1 | 391.9 | 962.6 | 1491.2 | 224.6 | 407.9 | 288.4 | 731.5 |
| YWHAQ | 16397.3 | 14896.6 | 25144.7 | 17152.0 | 19993.5 | 25095.3 | 21156.7 | 15419.7 | 18958.5 | 16735.7 | 18653.9 |
| TARDBP | 13101.6 | 12569.4 | 15698.2 | 10856.7 | 11763.0 | 12039.3 | 13220.1 | 14667.0 | 12740.1 | 12966.8 | 11231.6 |
| ADRB2 | 2984.9 | 2445.4 | 5693.0 | 2934.6 | 3250.7 | 5707.7 | 4771.8 | 3147.8 | 3012.0 | 3712.0 | 3256.7 |
| PPP1R8 | 2812.5 | 3028.8 | 3800.7 | 3148.1 | 3261.4 | 3183.9 | 3587.8 | 3395.0 | 3264.4 | 2999.5 | 3136.4 |
| MMAA | 213.7 | 210.0 | 333.4 | 239.1 | 259.3 | 311.1 | 281.2 | 260.9 | 250.3 | 261.0 | 276.5 |
| SQLE | 794.4 | 732.0 | 1039.7 | 790.1 | 782.8 | 695.1 | 741.1 | 1040.3 | 1134.3 | 840.0 | 722.6 |
| PDHA1 | 4186.6 | 4022.7 | 5019.7 | 3663.1 | 4354.7 | 5008.0 | 5101.1 | 4987.6 | 4731.1 | 4454.6 | 4853.7 |
| HAVCR2 | 2526.4 | 1731.1 | 2427.0 | 3492.6 | 2019.9 | 3318.7 | 5029.9 | 1772.0 | 2522.9 | 2037.5 | 4485.9 |
| RACGAP1 | 483.3 | 437.7 | 656.9 | 503.8 | 568.5 | 552.8 | 623.5 | 556.6 | 495.5 | 373.8 | 583.2 |
| AHNAK | 17327.5 | 13723.2 | 21366.6 | 13930.0 | 14121.5 | 16696.2 | 20000.5 | 14318.7 | 14420.7 | 14977.4 | 15971.5 |
| EDG8 | 724.6 | 322.9 | 987.9 | 626.7 | 559.9 | 1083.6 | 1187.5 | 225.8 | 429.0 | 285.7 | 684.1 |
| DUSP5 | 256.7 | 217.0 | 386.5 | 360.3 | 279.9 | 335.3 | 274.6 | 274.1 | 257.0 | 250.6 | 379.0 |

TABLE 17

| | Healthy individual | | | | | | | | | | | |
|---|---|---|---|---|---|---|---|---|---|---|---|---|
| | 111 | 112 | 113 | 114 | 115 | 116 | 117 | 118 | 119 | 120 | 121 | 122 |
| | Age | | | | | | | | | | | |
| | 77 | 67 | 67 | 67 | 67 | 60 | 68 | 31 | 35 | 32 | 31 | 31 |
| Gene | Sex | | | | | | | | | | | |
| symbol | Female | Female | Female | Female | Female | Female | Female | Male | Male | Male | Female | Female |
| FASLG | 986.3 | 1039.4 | 487.0 | 1027.8 | 583.6 | 1546.5 | 542.5 | 999.7 | 564.3 | 394.3 | 734.7 | 546.9 |
| CX3CR1 | 41655.5 | 28563.4 | 26816.6 | 48985.2 | 39931.3 | 50212.5 | 27259.8 | 51932.7 | 27195.9 | 39064.8 | 33768.3 | 23706.3 |
| TBX21 | 14415.6 | 11684.7 | 7059.6 | 12747.4 | 7133.7 | 18093.8 | 6309.8 | 20113.9 | 8482.5 | 10996.0 | 10757.3 | 4897.4 |
| ID2 | 5993.9 | 5078.5 | 3907.8 | 5483.3 | 3650.2 | 8080.1 | 4368.3 | 6395.2 | 4581.5 | 4043.4 | 5371.9 | 3731.5 |
| SLAMF7 | 4640.8 | 4303.6 | 2853.8 | 5545.3 | 4632.5 | 6007.1 | 3518.0 | 5513.4 | 2517.7 | 2895.8 | 3639.1 | 2749.1 |
| PRSS23 | 587.2 | 622.0 | 278.8 | 793.1 | 253.8 | 688.2 | 270.7 | 380.4 | 145.8 | 277.1 | 373.0 | 134.0 |
| YWHAQ | 17056.4 | 16796.8 | 15480.3 | 20745.5 | 17828.0 | 20406.7 | 18328.8 | 19399.0 | 14554.8 | 14704.4 | 14159.7 | 11936.0 |
| TARDBP | 12163.9 | 12598.1 | 12310.9 | 12867.2 | 11672.5 | 13429.2 | 13405.3 | 13213.0 | 13171.8 | 13931.7 | 11465.8 | 11619.0 |
| ADRB2 | 3138.2 | 4475.7 | 1872.9 | 3429.8 | 3110.9 | 4121.2 | 2311.6 | 4409.0 | 2546.7 | 2710.3 | 2351.1 | 2823.8 |
| PPP1R8 | 3499.9 | 3455.2 | 3045.9 | 3607.6 | 2434.6 | 4011.7 | 3139.5 | 2585.3 | 3122.2 | 2289.8 | 2840.7 | 2861.2 |
| MMAA | 230.9 | 258.7 | 253.0 | 289.2 | 231.8 | 266.9 | 261.5 | 226.0 | 224.7 | 188.5 | 211.9 | 192.0 |
| SQLE | 711.7 | 643.7 | 778.5 | 1028.3 | 1023.5 | 1050.2 | 656.6 | 689.9 | 651.1 | 669.0 | 596.0 | 1323.0 |
| PDHA1 | 4821.7 | 5351.2 | 4707.6 | 4849.4 | 4264.4 | 5627.8 | 4751.4 | 5355.3 | 5119.1 | 5135.9 | 4262.7 | 4265.9 |
| HAVCR2 | 2800.3 | 2300.9 | 2052.8 | 2186.2 | 1786.5 | 2682.9 | 2019.4 | 1995.6 | 1986.3 | 1559.3 | 2772.0 | 1915.2 |
| RACGAP1 | 551.0 | 476.9 | 440.9 | 482.0 | 362.8 | 588.4 | 515.5 | 369.1 | 430.3 | 402.9 | 384.9 | 413.7 |
| AHNAK | 14811.4 | 15390.9 | 11380.2 | 18778.5 | 15200.6 | 16576.1 | 12134.8 | 14037.3 | 14299.1 | 13870.4 | 9584.9 | 11800.7 |
| EDG8 | 574.9 | 572.5 | 278.7 | 523.3 | 289.4 | 813.5 | 237.1 | 797.2 | 267.6 | 263.7 | 522.6 | 234.6 |
| DUSP5 | 310.3 | 185.7 | 227.6 | 303.8 | 266.0 | 354.8 | 259.8 | 198.7 | 203.5 | 136.6 | 167.2 | 136.8 |

The analysis demonstrated that a healthy individual could be distinguished from a patient by analyzing the expression levels of FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5 in mRNA extracted from the peripheral whole blood of the subject, determining the mean expression level thereof, and using the mean as an indicator.

Also, all p values of genes shown in Table 2 were 0.000004 or smaller. Thus, the expression level of a single gene sufficiently enables distinguishing of a healthy individual from a patient. This indicates that depression can be diagnosed by measuring the expression level of at least one gene selected from among FASLG; CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5 in mRNA extracted from the peripheral whole blood of the subject, and determining whether or not a subject suffers from depression based on the expression level.

### Example 4

The expression levels of 18 genes described in Example 3 were used as indicators to perform diagnosis using a support vector machine. The sensitivity and the specificity in such a case were evaluated by the leave-one-out method. The results are shown in Table 18.

TABLE 18
Results of evaluation by the leave-one-out method
using support vector machine

| | Depressed patients | Healthy individuals |
|---|---|---|
| Diagnosed as “depression” | 38 | 10 |
| Diagnosed as “healthy” | 8 | 112 |

Sensitivity: 38/(38 + 8) = 82.6%
Specificity: 112/(10 + 112) = 91.8%
Accuracy: (38 + 112)/(38 + 10 + 8 + 112) = 89.3%

The results demonstrate that a depressed patient can be satisfactorily distinguished from a healthy individual with sensitivity of about 83%, specificity of about 92%, and accuracy of about 89%. This indicates that depression can be diagnosed by determining the expression levels of FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5 in mRNA extracted from the peripheral whole blood of the subjects, and determining whether or not a subject suffers from depression based on the results obtained with the use of a support vector machine.

### Example 5

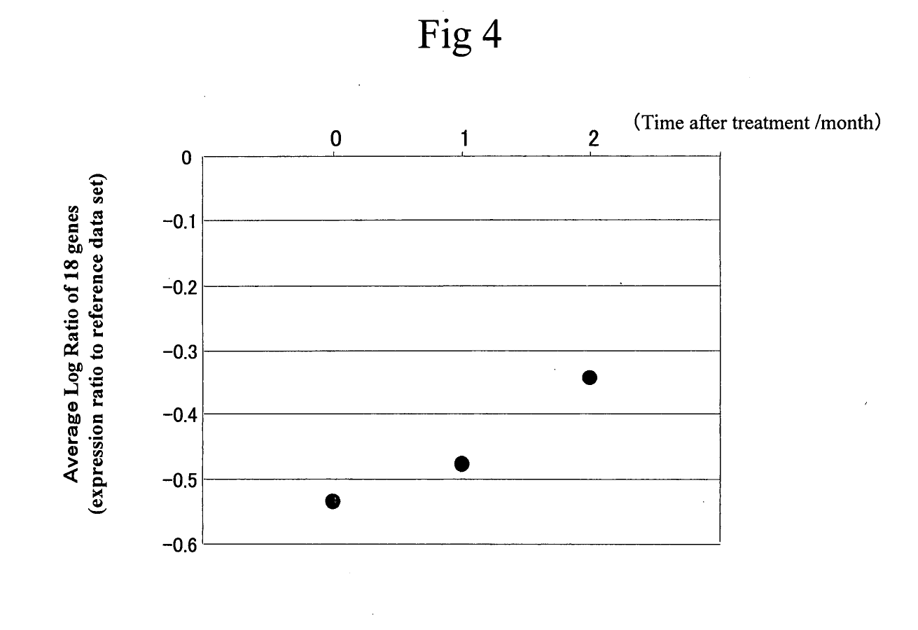

The results of diagnosis of depression with the elapse of treatment time were then examined. In addition to the patients before treatment, the blood samples obtained from 32 patients one month after the initiation of drug treatment and from seven patients two months after the initiation of drug treatment were subjected to gene expression analysis. The mean expression level of the 18 genes of the patients and that of the healthy individuals (i.e., the reference dataset) were determined, the mean Log value for the former mean to the latter mean were determined and plotted in FIG. 4. With the elapse of treatment time, the HAMD values were improved from 19.8 before treatment to 7.8 one month later and to 5.3 two months later as the symptom of depression is ameliorated, and the expression levels of the 18 genes were also increased uniformly (recovered to the level of a healthy individual).

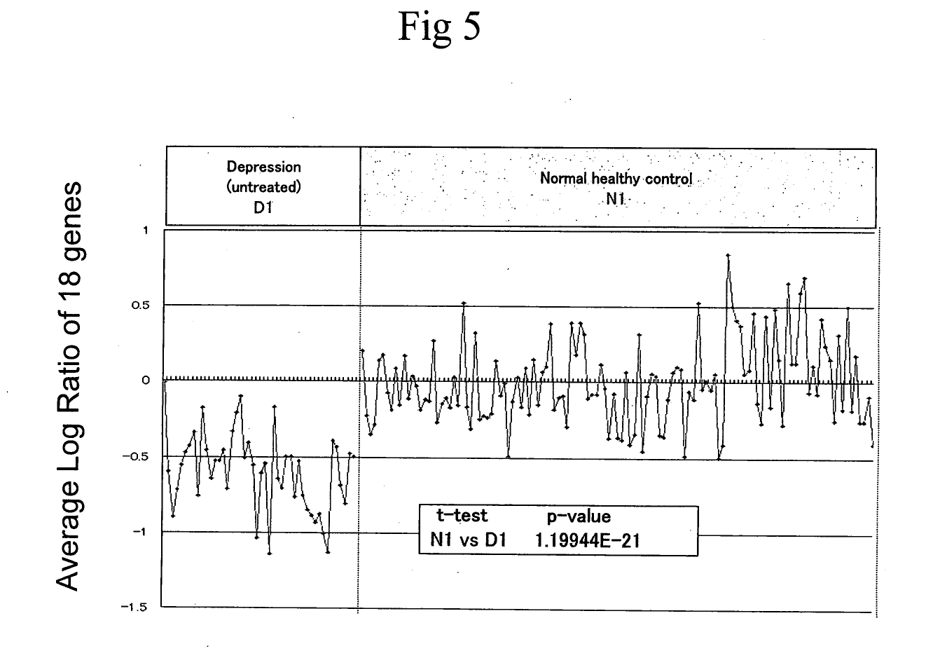

Further, the mean expression levels of the 18 genes (i.e., the mean Log ratio) of patients afflicted with depression before treatment (D1) and of healthy individuals (N1) were plotted in FIG. 5. The mean expression levels of the 18 genes are significantly different between patients afflicted with depression and healthy individuals (p value 1.19944 E-21).

The results indicate that the mean expression levels of the 18 genes can function not only as the indicators for distinguishing a healthy individual from a patient but also as the effective indicators for evaluating the effects of treatment. This demonstrates that gene expression analysis that is performed in the method of diagnosing depression enables inspection of therapeutic effects on depression.

### Example 6

Blood (5 ml each) was collected from healthy individuals (6 in total; 1 male and 1 female each in their 30's, 40's, and 50's) using a PAXgene Blood RNA System (Qiagen), and total RNA was extracted. The yield of total RNA was 5 μg to 15 μg. The quality of total RNA extracted from the subjects was inspected using the Bioanalyzer 2100 (Agilent) to confirm that total RNA had not been decomposed. Total RNA (0.2 mg) was then subjected to the in vitro transcription reaction to synthesize cRNA into which aminoallyl-CTP had been introduced in the presence of aminoallyl-CTP using the Agilent reagent that amplifies and synthesizes cRNA (Low RNA Input Linear Amp Kit PLUS, One-Color). Subsequently, an amino group in the synthesized cRNA was subjected to coupling to a succinimide-containing fluorescent dye (Cy3, Amersham) to synthesize fluorescent-labeled RNA. Subsequently, the fluorescent-labeled RNA was subjected to hybridization to the Agilent microarrays (Whole Human Genome Microarray 4 Pack) at 65° C. for 17 hours. The microarrays were washed in accordance with the Agilent's given protocols, and the fluorescent image was read using the Agilent scanner (Agilent). The image data was converted into the numerical data using a special software, Feature Extraction (Agilent). Subsequently, normalization was carried out so that a sum of signal intensities of genes exhibiting signal intensities of 25% to 75% would be 11, 234, and 345.

Target patients were as follows. Diagnosis was made in accordance with depressive episode specified in the International Classification of Diseases, 10th revision (ICD-10). Patients with serious physical complications or those taking therapeutic agents for physical diseases were excluded. Six patients whose samples before treatment had been obtained were 3 males and 3 females aged 38 to 55 (44 years old on average), and their Hamilton scores were from 17 to 31 (mean: 25.2). Blood (5 ml each) was collected from patients using a PAXgene Blood RNA System (Qiagen), and total RNA was extracted. The yield of total RNA was 5 μg to 15 μg. The quality of total RNA extracted from the patients was inspected using the Bioanalyzer 2100 (Agilent) to confirm that total RNA had not been decomposed. Total RNA (0.2 mg) was then subjected to the in vitro transcription reaction to synthesize cRNA into which aminoallyl-CTP had been introduced in the presence of aminoallyl-CTP using the Agilent reagent that amplifies and synthesizes cRNA (Low RNA Input Linear Amp Kit PLUS, One-Color). Subsequently, an amino group in the synthesized cRNA was subjected to coupling to a succinimide-containing fluorescent dye (Cy3, Amersham) to synthesize fluorescent-labeled RNA. Subsequently, the fluorescent-labeled RNA was subjected to unicolor hybridization to the Agilent microarrays (Whole Human Genome Microarray 4 Pack) at 65° C. for 17 hours. The microarrays were washed in accordance with the Agilent's given protocols, and the fluorescent image was read using the Agilent scanner (Agilent). The image data was converted into the numerical data using a special software, Feature Extraction (Agilent). Subsequently, normalization was carried out so that a sum of signal intensities of genes exhibiting signal intensities of 25% to 75% would be 11, 234, and 345.

As the indicators of diagnosis, FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5 genes were used, and expression levels thereof were determined. As the reference dataset, the mean dataset of 122 healthy individuals used in Examples 1 was used, and the data of the subjects were each divided by the reference data to determine the expression ratio. The databases of the group of patients and of the group of healthy individuals and the support vector machine-based diagnosis program prepared by the method described in Example 4 were used, and the data regarding the expression levels of six healthy individuals and six patients were applied as the query to the support vector machine-based software to diagnosis the subjects. The results are shown in Table 19.

TABLE 19
Results of evaluation in Example 6

| | Depressed patients | Healthy individuals |
|---|---|---|
| Diagnosed as “depression” | 5 | 0 |
| Diagnosed as “healthy” | 1 | 6 |

Sensitivity: 5/(5 + 1) = 83.3%
Specificity: 6/(0 + 6) = 100%
Accuracy: (5 + 6)/(5 + 1 + 0 + 6) = 91.7%

The results shown in Table 19 demonstrate that a depressed patient can be satisfactorily distinguished from a healthy individual with sensitivity of about 83.3%, specificity of 100%, and accuracy of about 91.7%.

Thus, diagnosis of depression via analysis of expression of a given group of genes was satisfactorily consistent with the results attained by clinical observation. This indicates that effects of the present invention are very high.

### Example 7

The expression levels of 9 genes of the 18 genes described in Example 3 (i.e., FASLG, CX3CR1, TBX21, ID2, SLAMF7, PRSS23, YWHAQ, TARDBP, and ADRB2 genes) were used as indicators to perform diagnosis using a support vector machine. The sensitivity and the specificity in such a case were evaluated by the leave-one-out method. The results are shown below.

TABLE 20
Results of evaluation in Example 7

| | Depressed patients | Healthy individuals |
|---|---|---|
| Diagnosed as “depression” | 36 | 22 |
| Diagnosed as “healthy” | 10 | 100 |

Sensitivity: 36/(36 + 10) = 78.3%
Specificity: 100/(22 + 100) = 81.1%
Accuracy: (36 + 100)/(36 + 10 + 22 + 100) = 81.0%

### Example 8

The expression levels of 7 genes of the 18 genes described in Example 3 (i.e., FASLG, CX3CR1, ID2, YWHAQ, TARDBP, EDG8, and DUSP5 genes) were used as indicators to perform diagnosis using a support vector machine. The sensitivity and the specificity in such a case were evaluated by the leave-one-out method. The results are shown below.

TABLE 21
Results of evaluation in Example 8

| | Depressed patients | Healthy individuals |
|---|---|---|
| Diagnosed as “depression” | 36 | 19 |
| Diagnosed as “healthy” | 10 | 103 |

Sensitivity: 36/(36 + 10) = 78.3%
Specificity: 103/(19 + 103) = 84.4%
Accuracy: (36 + 103)/(36 + 10 + 19 + 103) = 82.7%

### Example 9

The expression levels of 5 genes of the 18 genes described in Example 3 (i.e., FASLG, CX3CR1, ID2, YWHAQ, and TARDBP genes) were used as indicators to perform diagnosis using a support vector machine. The sensitivity and the specificity in such a case were evaluated by the leave-one-out method. The results are shown below.

TABLE 22
Results of evaluation in Example 9

| | Depressed patients | Healthy individuals |
|---|---|---|
| Diagnosed as “depression” | 32 | 19 |
| Diagnosed as “healthy” | 14 | 103 |

Sensitivity: 32/(32 + 14) = 70.0%
Specificity: 103/(19 + 103) = 84.4%
Accuracy: (32 + 103)/(32 + 14 + 19 + 103) = 80.4%

### Example 10

The expression levels of 11 genes of the 18 genes described in Example 3 (i.e., TBX21, SLAMF7, PRSS23, ADRB2, PPP1R8, MMAA, SQLE, PDHA1, HAVCR2, RACGAP1, and AHNAK genes) were used as indicators to perform diagnosis using a support vector machine. The sensitivity and the specificity in such a case were evaluated by the leave-one-out method. The results are shown below.

TABLE 23
Results of evaluation in Example 10

| | Depressed patients | Healthy individuals |
|---|---|---|
| Diagnosed as “depression” | 37 | 27 |
| Diagnosed as “healthy” | 9 | 95 |

Sensitivity: 37/(37 + 9) = 80.4%
Specificity: 95/(27 + 95) = 77.9%
Accuracy: (37 + 95)/(37 + 9 + 27 + 95) = 78.6%

The results obtained in Examples 7 to 10 demonstrate that accuracy of 70% or higher can be realized even when only some of the 18 genes are used. The accuracy for diagnosis of depression by a physician who is not specialized in depression is approximately 20% to 40%. Thus, the method for diagnosing depression according to the embodiments of the present invention was found to be useful as a tool for supporting diagnosis of depression. The method of the present invention can provide very useful information to a specialized physician as reference information for diagnosing a patient.

In the above embodiments, depression was diagnosed with the use of the reference dataset determined by the expression levels of healthy individuals, although a method for diagnosing depression is not limited thereto. For example, the expression levels of genes of many healthy individuals and/or the expression levels of the genes of many patients may be used to determine the cut-off values for the expression levels of the genes used for diagnosis, selected from among the 18 genes, and the determined values may be compared with the cut-off values of the expression levels of genes of the subjects. Thus, depression can be diagnosed.

Alternatively, the expression levels of genes of many healthy individuals and/or the expression levels of the genes of many patients may be used to determine the reference values for the expression levels of the genes used for diagnosis, selected from among the 18 genes, the ratio of the expression levels of the genes of target subjects to the reference levels of the genes is determined, and the determined ratio may be compared with the preset cut-off values. Thus, depression can be diagnosed.

Also, the expression levels of genes of many healthy individuals and/or the expression levels of the genes of many patients may be used to determine the cut-off values for the mean expression levels of the genes used for diagnosis among the 18 genes, and the expression levels of the genes of the target subjects may be compared with the cut-off values. Thus, depression can be diagnosed.

### INDUSTRIAL APPLICABILITY

The present invention has been completed based on the results of experiment that has analyzed gene expression in the peripheral whole blood sample of a depressed patient. With the use of the diagnostic method of the present invention, depression can be diagnosed in a simple manner with high accuracy.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

### LIST OF REFERENCES

1: Kawai T, Rokutan K. et al., Clin J Sport Med. 2007 September; 17 (5): 375-83, “Physical exercise-associated gene expression signatures in peripheral blood.”
2: Kawai T, Rokutan K., Biol Psychol. 2007 October; 76(3): 147-55. Epub 2007 Jul. 31, “Gene expression signature in peripheral blood cells from medical students exposed to chronic psychological stress.”
3: Omori T, Rokutan K., Seishin Shinkeigaku Zasshi. 2006; 108 (6): 642-5., “DNA microarray as a novel diagnostic marker for patients afflicted with depression”
4: Ohmori T, Rokutan K et al., J Med Invest. 2005 November; 52 Suppl: 266-71., “Assessment of human stress and depression by DNA microarray analysis.”
5: Morita K, Rokutan K. et al., Neurosci Lett. 2005 Jun. 10-17; 381(1-2):57-62. Epub 2005 Feb. 16., “Expression analysis of psychological stress-associated genes in peripheral blood leukocytes.”

### FREE TEXT OF SEQUENCE LISTING

### SEQ ID NO: 1: FASLG (GenBank Accession NO: NM_{—}000639)

### SEQ ID NO: 2: CX3CR1 (GenBank Accession NO: NM_{—}001337)

### SEQ ID NO: 3: TBX21 (GenBank Accession NO: NM_{—}013351)

### SEQ ID NO: 4: ID2 (GenBank Accession NO: NM_{—}002166)

### SEQ ID NO: 5: SLAMF7 (GenBank Accession NO: NM-021181)

### SEQ ID NO: 6: PRSS23 (GenBank Accession NO: NM_{—}007173)

### SEQ ID NO: 7: YWHAQ (GenBank Accession NO: NM_{—}006826)

### SEQ ID NO: 8: TARDBP (GenBank Accession NO: NM_{—}007375)

### SEQ ID NO: 9: ADRB2 (GenBank Accession NO: NM_{—}000024)

### SEQ ID NO: 10: PPP1R8 (GenBank Accession NO: NM-138558)

### SEQ ID NO: 11: MMAA (GenBank Accession NO: NM_{—}172250)

### SEQ ID NO: 12: SQLE (GenBank Accession NO: NM_{—}003129)

### SEQ ID NO: 13: PDHIA1 (GenBank Accession NO: NM_{—}000284)

### SEQ ID NO: 14: HAVCR2 (GenBank Accession NO: NM_{—}032782)

### SEQ ID NO: 15: RACGAP 1 (GenBank Accession NO: NM_{—}013277)

### SEQ ID NO: 16: AHNAK (GenBank Accession NO: NM_{—}001620)

### SEQ ID NO: 17: EDG8 (GenBank Accession NO: NM_{—}030760)

### SEQ ID NO: 18: DUSP5 (GenBank Accession NO: NM_{—}004419)

Sequence Listing

What is claimed is:

1. A method for diagnosing depression comprising the steps of:
measuring expression levels of 18 genes selected from the group consisting of FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5, in peripheral blood isolated from a subject; and
determining whether or not the subject suffers from depression based on the expression levels of the 18 genes.

2. The method according to claim 1, comprising the step of comparing the expression levels of the 18 genes of the subject with expression levels of the genes of healthy individual, wherein the determining step is performed by determining whether or not the subject suffers from depression based on the comparison result.

3. The method according to claim 1, comprising the steps of:
obtaining an expression ratio of the expression levels of the 18 genes of the subject to expression levels of the genes of healthy individuals; and
comparing the expression ratio with predetermined data concerning expression levels of the genes of a depressed patient and of healthy individual, wherein the determining step is performed by determining whether or not the subject suffers from depression based on the comparison result.

4. The method according to claim 3, wherein the comparing step is carried out using a support vector machine.

5. The method according to claim 1 comprising the steps of:
obtaining a mean value of the expression levels of the 18 genes of the subject; and
comparing the mean expression level with a mean expression level of the 18 genes of healthy individual, wherein the determining step is performed by determining whether or not the subject suffers from depression based on the comparison result.

6. The method according to claim 5, wherein the determining step is performed by determining that the subject has a high possibility of suffering from depression when the mean expression level of the 18 genes of the subject is significantly lower than that of the healthy individual.

7. The method according to claim 1, wherein the gene expression levels of the 18 genes are measured with the use of nucleic acid-immobilized solid substrates, such as a DNA chip or an array.

8. A method for diagnosing depression comprising the steps of:
measuring expression level of at least one of 18 genes selected from the group consisting of FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5, in peripheral blood isolated from a subject; and
determining whether or not the subject suffers from depression based on the expression level.

9. The method according to claim 8, comprising the step of comparing the expression level of at least one of the 18 genes of the subject with a predetermined reference value, wherein the determining step is performed by determining whether or not the subject suffers from depression based on the comparison results.

10. The method according to claim 8, comprising the steps of:
obtaining a ratio of the expression level of at least one of the 18 genes of the subject to a predetermined reference value; and
comparing the ratio with a predetermined threshold value, wherein the determining step is performed by determining whether or not the subject suffers from depression based on the comparison results.

11. The method according to claim 8, comprising the steps of:
obtaining a mean value of the expression levels of at least two of the 18 genes of the subject; and
comparing the mean value with a predetermined threshold value, wherein the determining step is performed by determining whether or not the subject suffers from depression based on the comparison results.

12. The method according to claim 11, wherein the determining step is performed by determining that the subject has a high possibility of suffering from depression when the mean value of at least two of the 18 genes of the subject is lower than the threshold value.

13. A program for performing the method for diagnosing depression comprising:
1) a means for inputting expression levels of 18 genes selected from the group consisting of FASLG, CX3CR1, TBX21, ID2, SLAMF7, PRSS23, YWHAQ, TARDBP, ADRB2, PPP1R8, MMAA, SQLE, PDHA1, HAVCR2, RACGAP1, AHNAK, EDG8, and DUSP5, in the peripheral blood isolated from a subject;
2) a means for storing data concerning expression levels of the 18 genes of depressed patient and of healthy individual that have been inputted in advance;
3) a means for comparing the expression levels of the 18 genes of the subject with the expression levels of the 18 genes of healthy individual;
4) a means for determining whether or not the subject suffers from depression based on the results of comparison; and
5) a means for outputting the results of determination.

14. The program according to claim 13, wherein in 3) above, an expression ratio of the expression levels of the 18 genes of the subject to those of healthy individual is obtained in order to compare said expression ratio with an expression ratio of expression levels of the 18 genes of depressed patient to expression levels of the 18 genes of healthy individual that have been stored in advance.

15. The program according to claim 14, wherein the analysis is carried out using a support vector machine.

16. The program according to claim 13, wherein in 3) above, the mean value of the expression levels of the 18 genes of the subject is obtained, and the mean value is compared with a mean value of expression levels of the 18 genes of healthy individual.

17. The program according to claim 13, further comprising a means for storing the expression levels of the 18 genes of the subject and updating the data of the depressed patient and that of healthy individual according to need.


SEQUENCE LISTING (18 sequences)

SEQ ID NO: 1 - DNA, 1909 bp, Homo sapiens
  GenBank ID NM_000639
gaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaaaaaaaaaaaaaaaaaaaaaaaaaaaaaaaaaaaaaaaaaaaaaaaaaaa

SEQ ID NO: 2 - DNA, 3108 bp, Homo sapiens
  GenBank ID NM_001337
gaaatactcgtctctggtaaagtctgagcaggacagggtggctgactggcagatccagaggttcccttggcagtccacgccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttcca

SEQ ID NO: 3 - DNA, 2589 bp, Homo sapiens
  GenBank ID NM_013351
cggcccgctggagaggaagcccgagagctgccgcgcgcctgccggacgagggcgtagaagccaggcgtcagagcccgggctccggtggggtcccccacccggccctcgggtcccccgccccctgctccctgcccatcccagcccacgcgaccctctcgcgcgcggaggggcgggtcctcgacggctacgggaaggtgccagcccgccccggatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactgagcagatgacatgatgaaaggaacagaaacagtgttattaggttggaggacaccgactaatttgggaaacggatgaaggactgagaaggcccccgctccctctggcccttctctgtttagtagttggttggggaagtggggctcaagaaggattttggggttcaccagatgcttcctggcccacgatgaaacctgagaggggtgtccccttgccccatcctctgccctaactacagtcgtttacctggtgctgcgtcttgcttttggtttccagctggagaaaagaagacaagaaagtcttgggcatgaaggagctttttgcatctagtgggtgggaggggtcaggtgtgggacatgggagcaggagactccactttcttcctttgtacagtaactttcaaccttttcgttggcatgtgtgttaatccctgatccaaaaagaacaaatacacgtatgttataaccatcagcccgccagggtcagggaaaggactcacctgactttggacagctggcctgggctccccctgctcaaacacagtggggatcagagaaaaggggctggaaaggggggaatggcccacatctcaagaagcaagatattgtttgtggtggttgtgtgtgggtgtgtgttttttctttttctttctttttattttttttgaatgggggaggctatttattgtactgagagtggtgtctggatatattccttttgtcttcatcactttctgaaaataaacataaaactgttaaaaaaaaaaaaaaaaa

SEQ ID NO: 4 - DNA, 1402 bp, Homo sapiens
  GenBank IDNM_002166
ggggacgaagggaagctccagcgtgtggccccggcgagtgcggataaaagccgccccgccgggctcgggcttcattctgagccgagcccggtgccaagcgcagctagctcagcaggcggcagcggcggcctgagcttcagggcagccagctccctcccggtctcgccttccctcgcggtcagcatgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctgaataagcggtgttcatgatttcttttattctttgcacaacaacaacaacaacaaattcacggaatcttttaagtgctgaacttatttttcaaccatttcacaaggaggacaagttgaatggacctttttaaaaagaaaaaaaaaatggaaggaaaactaagaatgatcatcttcccagggtgttctcttacttggactgtgatattcgttatttatgaaaaagacttttaaatgccctttctgcagttggaaggttttctttatatactattcccaccatggggagcgaaaacgttaaaatcacaaggaattgcccaatctaagcagactttgccttttttcaaaggtggagcgtgaataccagaaggatccagtattcagtcacttaaatgaagtcttttggtcagaaattacctttttgacacaagcctactgaatgctgtgtatatatttatatataaatatatctatttgagtgaaaccttgtgaactctttaattagagttttcttgtatagtggcagagatgtctatttctgcattcaaaagtgtaatgatgtacttattcatgctaaactttttataaaagtttagttgtaaacttaacccttttatacaaaataaatcaagtgtgtttattgaatggtgattgcctgctttatttcagaggaccagtgctttgatttttattatgctatgttataactgaacccaaataaatacaagttcaaatttatgtagactgtataagattataataaaacatgtctgaagtcaaaaaaaaaaaaaaaaaaaaaaaaaaaaaaaaaaaaaaaa

SEQ ID NO: 5 - DNA, 2672 bp, Homo sapiens
  GenBank IDNM_021181
cttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctatagaaatgtaaatgcaaggtcacacatattaatgacagcctgttgtattaatgatggctccaggtcagtgtctggagtttcattccatcccagggcttggatgtaaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacgtgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaacacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcaaaaaaaaaaaaaaaa

SEQ ID NO: 6 - DNA, 3806 bp, Homo sapiens
  GenBank IDNM_007173
gcggcttccccgaggccggaggcggggcgggcgggcctcgggtggcgcggggggcggacccgccagctgcctgcgctgctcgccagcttgctcgcactcggctgtgcggcggggcaggcatgggagccgcgcgctctctcccggcgcccacacctgtctgagcggcgcagcgagccgcggcccgggcgggctgctcggcgcggaacagtgctcggcatggcagggattccagggctcctcttccttctcttctttctgctctgtgctgttgggcaagtgagcccttacagtgccccctggaaacccacttggcctgcataccgcctccctgtcgtcttgccccagtctaccctcaatttagccaagccagactttggagccgaagccaaattagaagtatcttcttcatgtggaccccagtgtcataagggaactccactgcccacttacgaagaggccaagcaatatctgtcttatgaaacgctctatgccaatggcagccgcacagagacgcaggtgggcatctacatcctcagcagtagtggagatggggcccaacaccgagactcagggtcttcaggaaagtctcgaaggaagcggcagatttatggctatgacagcaggttcagcatttttgggaaggacttcctgctcaactaccctttctcaacatcagtgaagttatccacgggctgcaccggcaccctggtggcagagaagcatgtcctcacagctgcccactgcatacacgatggaaaaacctatgtgaaaggaacccagaagcttcgagtgggcttcctaaagcccaagtttaaagatggtggtcgaggggccaacgactccacttcagccatgcccgagcagatgaaatttcagtggatccgggtgaaacgcacccatgtgcccaagggttggatcaagggcaatgccaatgacatcggcatggattatgattatgccctcctggaactcaaaaagccccacaagagaaaatttatgaagattggggtgagccctcctgctaagcagctgccagggggcagaattcacttctctggttatgacaatgaccgaccaggcaatttggtgtatcgcttctgtgacgtcaaagacgagacctatgacttgctctaccagcaatgcgatgcccagccaggggccagcgggtctggggtctatgtgaggatgtggaagagacagcagcagaagtgggagcgaaaaattattggcattttttcagggcaccagtgggtggacatgaatggttccccacaggatttcaacgtggctgtcagaatcactcctctcaaatatgcccagatttgctattggattaaaggaaactacctggattgtagggaggggtgacacagtgttccctcctggcagcaattaagggtcttcatgttcttattttaggagaggccaaattgttttttgtcattggcgtgcacacgtgtgtgtgtgtgtgtgtgtgtgtgtaaggtgtcttataatcttttacctatttcttacaattgcaagatgactggctttactatttgaaaactggtttgtgtatcatatcatatatcatttaagcagtttgaaggcatacttttgcatagaaataaaaaaaatactgatttggggcaatgaggaatatttgacaattaagttaatcttcacgtttttgcaaactttgatttttatttcatctgaacttgtttcaaagatttatattaaatatttggcatacaagagatatgaattcttatatgtgtgcatgtgtgttttcttctgagattcatcttggtggtgggtttttttgtttttttaattcagtgcctgatctttaatgcttccataaggcagtgttcccatttaggaactttgacagcatttgttaggcagaatattttggatttggaggcatttgcatggtagtctttgaacagtaaaatgatgtgttgactatactgatacacatattaaactataccttatagtaaaccagtatcccaagctgcttttagttccaaaaatagtttcttttccaaaggttgttgctctactttgtaggaagtctttgcatatggccctcccaactttaaagtcataccagagtggccaagagtgtttatcccaacccttccatttaacaggatttcactcacatttctggaactagctatttttcagaagacaataatcagggcttaattagaacaggctgtatttcctcccagcaaacagttgtggccacactaaaaacaatcatagcattttacccctggattatagcacatctcatgttttatcatttggatggagtaatttaaaatgaattaaattccagagaacaatggaagcattgcctggcagatgtcacaacagaataaccacttgtttggagcctggcacagtcctccagcctgatcaaaaattattctgcatagttttcagtgtgctttctgggagctatgtacttcttcaatttggaaacttttctctctcatttatagtgaaaatacttggaagttactttaagaaaaccagtgtggcctttttccctctagctttaaaagggccgcttttgctggaatgctctaggttatagataaacaattaggtataatagcaaaaatgaaaattggaagaatgcaaaatggatcagaatcatgccttccaataaaggcctttacacatgttttatcaatatgattatcaaatcacagcatatacagaaaagacttggacttattgtatgtttttattttatggctctcggcctaagcacttctttctaaatgtatcggagaaaaaatcaaatggactacaagcacgtgtttgctgtgcttgcaccccaggtaaacctgcattgtagcaatttgtaaggatattcagatggagcactgtcacttagacattctctgggggattttctgcttgtctttcttgagctttttggaaggataattctgataaggcactcaagaaacgtacaaccacagtgctttcttcaaatcatatgagaaatactatgcatagcaaggagatgcagagccgccaggaaaattctgagttccagcacaattttctttggaatctaacaggaatctagcctgaggaagaagggaggtctccatttctatgtctggtatttgggggttttgtttgtttttgctttagcttggtgaaaaaaagttcactgaacaccaagaccagaatggatttttttaaaaaaatagatgttccttttgtgaagcaccttgattccttgattttgattttttgcaaagttagacaatggcacaaagtcaaaatgaaatcaatgtttagttcacaagtagatgtaatttactaaagaatgatacacccatatgctatatacagcttaactcacagaactgtaaaagaaaattataaaataattcaacatgtccatctttttagtgataataaaagaaagcatggtattaaactatcatagaagtagacagaaaaagaaaaaaggactcatggcattattaatataattagtgctttacatgtgttagttatacatattagaagcatatttgcctagtaaggctagtagaaccacatttcccaaagtgtgctccttaaacactcatgccttatgattttctaccaaaagtaaaaagggttgtattaagtcagaggaagatgcctctccattttccctctctttatcagaggttcacatgcctgtctgcacattaaaagctctgggaagacctgttgtaaagggacaagttgaggttgtaaaatctgcatttaaataaacatctttgatcacaaaa

SEQ ID NO: 7 - DNA, 2166 bp, Homo sapiens
  GenBank IDNM_006826
gtggtgggactcgcgtcgcggccgcggagacgtgaagctctcgaggctcctcccgctgcgggtcggcgctcgccctcgctctcctcgccctccgccccggccccggccccgcgcccgccatggagaagactgagctgatccagaaggccaagctggccgagcaggccgagcgctacgacgacatggccacctgcatgaaggcagtgaccgagcagggcgccgagctgtccaacgaggagcgcaacctgctctccgtggcctacaagaacgtggtcgggggccgcaggtccgcctggagggtcatctctagcatcgagcagaagaccgacacctccgacaagaagttgcagctgattaaggactatcgggagaaagtggagtccgagctgagatccatctgcaccacggtgctggaattgttggataaatatttaatagccaatgcaactaatccagagagtaaggtcttctatctgaaaatgaagggtgattacttccggtaccttgctgaagttgcgtgtggtgatgatcgaaaacaaacgatagataattcccaaggagcttaccaagaggcatttgatataagcaagaaagagatgcaacccacacacccaatccgcctggggcttgctcttaacttttctgtattttactatgagattcttaataacccagagcttgcctgcacgctggctaaaacggcttttgatgaggccattgctgaacttgatacactgaatgaagactcatacaaagacagcaccctcatcatgcagttgcttagagacaacctaacactttggacatcagacagtgcaggagaagaatgtgatgcggcagaaggggctgaaaactaaatccatacagggtgtcatccttctttccttcaagaaacctttttacacatctccattccttattccacttggatttcctatagcaaagaaacccattcatgtgtatggaatcaactgtttatagtcttttcacactgcagctttgggaaaacttcattccttgatttgtgtttgtcttggccttcctggtgtgcagtactgctgtagaaaagtattaatagcttcatttcatataaacataagtaactcccaaacacttatgtagaggactaaaaatgtatctggtatttaagtaatctgaaccagttctgcaagtgactgtgttttgtattactgtgaaaataagaaaatgtagttaattacaatttaaagagtattccacataacttcttaatttctacattccctcccttactcttcgggggtttcctttcagtaagcaacttttccatgctcttaatgtattcctttttagtaggaatccggaagtattagattgaatggaaaagcacttgccatctctgtctaggggtcacaaattgaaatggctcctgtatcacatacggaggtcttgtgtatctgtggcaacagggagtttccttattcactctttatttgctgctgtttaagttgccaacctcccctcccaataaaaattcacttacacctcctgcctttgtagttctggtattcactttactatgtgatagaagtagcatgttgctgccagaatacaagcattgcttttggcaaattaaagtgcatgtcatttcttaatacactagaaaggggaaataaattaaagtacacaagtccaagtctaaaactttagtacttttccatgcagatttgtgcacatgtgagagggtgtccagtttgtctagtgattgttatttagagagttggaccactattgtgtgttgctaatcattgactgtagtcccaaaaaagccttgtgaaaatgttatgccctatgtaacagcagagtaacataaaataaaagtacattttataaaccatttactatggctttgtaacaattgcatacccatattttaagggacaggtgaatttactactttctaaagtttattgatacttcccttttatgtaaaatgtagtagtgatacctatatttccacattgtgcattgtgacacacttgtctagggatgcctggaagtgtataaaattggactgcatttcttagagtgttttactatagatcagtctcatgggccatctcttcctcagatgtaaatgatatctggttaagtgttatatggaataaagtggacattttaaaacta

SEQ ID NO: 8 - DNA, 4236 bp, Homo sapiens
  GenBank IDNM_007375
ggtgggcggggggaggaggcggccctagcgccattttgtgggagcgaagcggtggctgggctgcgcttgggtccgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaatttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacataaacatttatcaatgaagtcatcctttagacttgtaatcgccacattgtttcattattcagtttcctctgtaaagggatcttgagttgttttaattttttttttctgcatctgaatctgcatgatttccaaaccctgtaccatctgaattttgcattttagcacttgcactattactcagcagcagtaacatggtaacacttaaaatggtactcggggacctccaaagactaaactgacaagccttcaaggagcccaggggtaagttaacttgtcaacggcatggtttaatcccttctttacacttgtgtaaatttcagttactggtcatagaaggctttcaatgttgagtggccttttattaacatgtttatggtactgcatagatacgggtatttattttaccctaagaagattttgaagtttaaaagtacttaaactatttggcaaagatttgtttttaaaaatctatttggtcaatctaaatgcattcattctaaaaaattttttgaaccagataaataaaatttttttttgacaccacaaaaaaaaaaaaaaaaaaaa

SEQ ID NO: 9 - DNA, 2033 bp, Homo sapiens
  GenBank ID NM_000024
gcacataacgggcagaacgcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccaacagaacactaaacagactatttaacttgagggtaataaacttagaataaaattgtaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaagtctaaagtttacagtaaataaattgtttgaccatgcc

SEQ ID NO: 10 - DNA, 2691 bp, Homo sapiens
  GenBank ID NM_138558
cttccagtttcccggcgtgcttagggcgcgccaaatgggagggggagacgcaagatggcggcagccgcgaactccggctctagcctcccgctgttcgactgcccaacctggtgagtggcggggcggccagggctagagtggcccggccggagctagcctgggctggaagggcggctctttttttacttttctgctgcgagccgaacggctcagaaaccccggaatggttgaggaaaaactgtttgctgcaccgggccgggcgacgtgttgaagaaccgagagcctggagcccaggcccaggaactgaagaaacccggggttgggggctcaaaggcgctcacttaggcagcccctttgagcgattagccagtcgccggagcgcttcgaggccttggcccgaacttacgcccaactcttgactgagtgcctggtgctctcgtggagcatcgcatctggccccttcctggcaggtaagccccctcccggtttacatctggatgtagtcaaaggagacaaactaattgagaaactgattattgatgagaagaagtattacttatttgggagaaaccctgatttgtgtgactttaccattgaccaccagtcttgctctcgggtccatgctgcacttgtctaccacaagcatctgaagagagttttcctgatagatctcaacagtacacacggcactttcttgggtcacattcggttggaacctcacaagcctcagcaaattcccatcgattccacggtctcatttggcgcatccacaagggcatacactctgcgcgagaagcctcagacattgccatcggctgtgaaaggagatgagaagatgggtggagaggatgatgaactcaagggcttactggggcttccagaggaggaaactgagcttgataacctgacagagttcaacactgcccacaacaagcggatttctacccttaccattgaggagggaaatctggacattcaaagaccaaagaggaagaggaagaactcacgggtgacattcagtgaggatgatgagatcatcaacccagaggatgtggatccctcagttggtcgattcaggaacatggtgcaaactgcagtggtcccagtcaagaagaagcgtgtggagggccctggctccctgggcctggaggaatcagggagcaggcgcatgcagaactttgccttcagcggaggactctacgggggcctgccccccacacacagtgaagcaggctcccagccacatggcatccatgggacagcactcatcggtggcttgcccatgccatacccaaaccttgcccctgatgtggacttgactcctgttgtgccgtcagcagtgaacatgaaccctgcaccaaaccctgcagtctataaccctgaagctgtaaatgaacccaagaagaagaaatatgcaaaagaggcttggccaggcaagaagcccacaccttccttgctgatttgatatttttggtcatggagaagggtgggattgggtgggaatggggtggaagggtgatggggagctaatgaactagggagaaaaactttccatgtgtgcggtatcgtctttcagaatgtctcctggcatcctaaccatgtaatatgacaattgggggtggggttgaaatagcccataaagacctgtcttcacaacacttgcattgtagagaaaggcttcttatatccttttcaatagactgccctggctctttcctaggccttccactacctcctttctttctcccactttctaggatcatttttatgtaaagtcacatatcccaggccctcaggttgaatccagagctgtagaggttacagtagcatcaccagccttgggggtccagagcctaatttatattcactatccttccaagtcccgggtagcagaagggttgccatagatctcagtttgatcaaaaagaaggcttagaattctgcagttaagctgaggtttaaactaaaaaatgtttccttgggtcagtggttttgaggtccagtagctaggcttttctcttttgtccttcctgttggaatgaaaacatttcgattttccttcatctgtgactggtgccatagacacaggtttatagttttaacttacagtattgtttgaaatttacctgtttttcttgtcaaacctgagcactcctcctgctgaagtttcttatttaattccagagtactgtcctctactctaaggcattacttttaagtgtattatgaaggcagttttcaaaggatatgaccagttggggtaattcaaattaaaaaggaaaagatttgtttggaagtaactggtgtctctaagaggaatttttagatgtcagtttggaggctctttcccccctcaattgagagctcttgttattcagagctccaagactagacctggctaacaaacataggagacaaagttaggaaacattgatacaagctttgtacagagatttgtacatttgtgtaataggccttttcatgctttatgtgtagctttttacctgtaacctttattacattgtaaattaaacgtaacttttgtcatttgggtgcaggctgtgaatttgtctctcagtcactgattgccactgccatctggaaatgtttgctaaaggcacagtcactgggcttgggaggcaatgctccatccccattatattacaaataaagatgccctaaatgagtgtg

SEQ ID NO: 11 - DNA, 1473 bp, Homo sapiens
  GenBank ID NM_172250
ggaggtcacaatcacattgagccaaaacgcatccagtgttttctccagttacaaataaaacgaatatgcccatgctgctaccacatcctcaccagcatttcctaaaaggccttttaagagcacctttccgatgttaccacttcatctttcactcaagtactcatctcggatcaggaatcccatgtgctcagccgtttaattctcttggactccattgtacaaagtggatgctgctgtcagatggcttaaagagaaaattatgtgtacaaacaaccttaaaggaccacacagaaggactttctgataaagagcaaagatttgtggataaactttatactggtttaatccaagggcaaagggcctgtttagcagaggccataactcttgtagaatcaactcacagcaggaaaaaggagttagcccaggtgcttcttcagaaagtattactttaccacagagaacaagaacaatcaaataaaggaaaaccactagcatttcgagtaggattgtctgggccccctggtgctggaaaatcaacatttatagaatattttggaaaaatgcttactgagagagggcacaaattatctgtgctagctgtggacccttcttcttgtactagtggtggatcactcttaggtgataaaacccgaatgactgagttatcaagagatatgaatgcatacatcaggccatctcctactagaggaactttaggaggcgtgacaaggaccacaaatgaagctattctgttgtgtgaaggagcgggatatgacataattcttattgaaaccgttggtgtgggtcagtcggagtttgctgttgctgacatggttgacatgtttgttttactactgccaccagcaggaggagatgagctgcagggtatcaaaaggggtataatcgagatggcagatctggtagctgtaactaaatctgatggagacttgattgtgccagctcgaaggatacaagcggaatatgtgagtgcactgaaattactccgcaaacgttcacaagtctggaaaccaaaggtaattcgtatttctgcccgaagtggagaggggatctctgaaatgtgggataaaatgaaagatttccaggacctaatgcttgccagtggggagctgactgccaaacgacggaagcaacagaaagtttggatgtggaatctcattcaggaaagtgtgttagagcatttcaggacccaccccacagtccgggaacagattccacttctggaacaaaaggttctcattggggccctgtccccaggactagcagcagacttcttgttaaaagcttttaaaagcagagactaataaaattcatcctgtataataattttacatatcatttcataaagtattttaatagaaaaatcacttgtatgcttatattttcagtaattattgtatggtgctcttgtcttctttgtttgtgacccatgcttgaaaacttgaaggaagttag

SEQ ID NO: 12 - DNA, 2989 bp, Homo sapiens
  GenBank ID NM_003129
gtctgggccgagcccgcccagctggctgagacgcgtggagcctggcggcgagtgggggcgtgcgacggttactctggttactggggccgcgccgcgctggcgagagccgccgcccgcgagggatgctggtgaggaagccgtcgggagccgccgccgccatctgagggaggtaccctggaaaccaccttttatcggtggggaagtgcagtcgcggtgggcggctctgggggccagcgaaacgggaggcctctaaatctttaggttggggctgcattgccctggagccgcactcttgagtccgaggccatcttttgttggagaaggcgtcggcgttggcgttttcccgaggttgggctgtacagtgtctccgtccgcggaaaaagaagcctctgaacccgcgccggcccgcagcccccgtgccttccggccgctgctcgccgtcgccagaggctaggccacgtttcccccagtgccgaggtgtttctgtgaccctccctccactcccattcccttctgaaagggcacctgctcttggtgagaaaagaaattatagcacgaagagccagtatcagaagagtatccatcacccgcagcaaccgctcagggaacaccatcaaaaaagaaaaaaagggaatatctggatttcctgggcgaggaggagcgagtctgctcgggagctgttccagcaggcgatttttaaatactgctttctacgccctatacaacttggcttcacatacttttacactaactttatatgatttttaaaaactggtctgatcggacttctcgtcctgggacactgtttactggagtctggccggctctccgtgctcctcttggtacctcattttggggagaaccttaaacccactcgagcagataatctccgccttgaccggtgccaccaaagaagccttggaaccatgtggacttttctgggcattgccactttcacctatttttataagaagttcggggacttcatcactttggccaacagggaggtcctgttgtgcgtgctggtgttcctctcgctgggcctggtgctctcctaccgctgtcgccaccgaaacgggggtctcctcgggcgccagcagagcggctcccagttcgccctcttctcggatattctctcaggcctgcctttcattggcttcttctgggccaaatccccccctgaatcagaaaataaggagcagctcgaggccaggaggcgcagaaaaggaaccaatatttcagaaacaagcttaataggaacagctgcctgtacatcaacatcttctcagaatgacccagaagttatcatcgtgggagctggcgtgcttggctctgctttggcagctgtgctttccagagatggaagaaaggtgacagtcattgagagagacttaaaagagcctgacagaatagttggagaattcctgcagccgggtggttatcatgttctcaaagaccttggtcttggagatacagtggaaggtcttgatgcccaggttgtaaatggttacatgattcatgatcaggaaagcaaatcagaggttcagattccttaccctctgtcagaaaacaatcaagtgcagagtggaagagctttccatcacggaagattcatcatgagtctccggaaagcagctatggcagagcccaatgcaaagtttattgaaggtgttgtgttacagttattagaggaagatgatgttgtgatgggagttcagtacaaggataaagagactggagatatcaaggaactccatgctccactgactgttgttgcagatgggcttttctccaagttcaggaaaagcctggtctccaataaagtttctgtatcatctcattttgttggctttcttatgaagaatgcaccacagtttaaagcaaatcatgctgaacttattttagctaacccgagtccagttctcatctaccagatttcatccagtgaaactcgagtacttgttgacattagaggagaaatgccaaggaatttaagagaatacatggttgaaaaaatttacccacaaatacctgatcacctgaaagaaccattcttagaagccactgacaattctcatctgaggtccatgccagcaagcttccttcctccttcatcagtgaagaaacgaggtgttcttcttttgggagacgcatataatatgaggcatccacttactggtggaggaatgactgttgcttttaaagatataaaactatggagaaaactgctaaagggtatccctgacctttatgatgatgcagctattttcgaggccaaaaaatcattttactgggcaagaaaaacatctcattcctttgtcgtgaatatccttgctcaggctctttatgaattattttctgccacagatgattccctgcatcaactaagaaaagcctgttttctttatttcaaacttggtggcgaatgtgttgcgggtcctgttgggctgctttctgtattgtctcctaaccctctagttttaattggacacttctttgctgttgcaatctatgccgtgtatttttgctttaagtcagaaccttggattacaaaacctcgagcccttctcagtagtggtgctgtattgtacaaagcgtgttctgtaatatttcctctaatttactcagaaatgaagtatatggttcattaagcttaaaggggaaccatttgtgaatgaatatttggaacttaccaagtcctaagagacttttggaagaggatatatatagcatagtaccataccacttataaagtggaaactcttggaccaagatttggattaatttgtttttgaagttttttgtatataaatatgtaaatacatgctttaatttgcaatttaaaatgaaggggttaaataagttagacatttaaaagaaatgattgttaccataaattagtgctaatgctgaggagaactacagtttttcttttgaatttagtatttgagatgagttgttgggacatgcaaataaaatgaagaatgaa

SEQ ID NO: 13 - DNA, 3364 bp, Homo sapiens
  GenBank ID NM_000284
agcgcatgacgttattacgactctgtcacgccgcggtgcgactgaggcgtggcgtctgctggggcacctgaaggagacttgggggcacccgcgtcgtgcctcctgggttgtgaggagtcgccgctgccgccactgcctgtgcttcatgaggaagatgctcgccgccgtctcccgcgtgctgtctggcgcttctcagaagccggcaagcagagtgctggtagcatcccgtaattttgcaaatgatgctacatttgaaattaagaaatgtgaccttcaccggctggaagaaggccctcctgtcacaacagtgctcaccagggaggatgggctcaaatactacaggatgatgcagactgtacgccgaatggagttgaaagcagatcagctgtataaacagaaaattattcgtggtttctgtcacttgtgtgatggtcaggaagcttgctgtgtgggcctggaggccggcatcaaccccacagaccatctcatcacagcctaccgggctcacggctttactttcacccggggcctttccgtccgagaaattctcgcagagcttacaggacgaaaaggaggttgtgctaaagggaaaggaggatcgatgcacatgtatgccaagaacttctacgggggcaatggcatcgtgggagcgcaggtgcccctgggcgctgggattgctctagcctgtaagtataatggaaaagatgaggtctgcctgactttatatggcgatggtgctgctaaccagggccagatattcgaagcttacaacatggcagctttgtggaaattaccttgtattttcatctgtgagaataatcgctatggaatgggaacgtctgttgagagagcggcagccagcactgattactacaagagaggcgatttcattcctgggctgagagtggatggaatggatatcctgtgcgtccgagaggcaacaaggtttgctgctgcctattgtagatctgggaaggggcccatcctgatggagctgcagacttaccgttaccacggacacagtatgagtgaccctggagtcagttaccgtacacgagaagaaattcaggaagtaagaagtaagagtgaccctattatgcttctcaaggacaggatggtgaacagcaatcttgccagtgtggaagaactaaaggaaattgatgtggaagtgaggaaggagattgaggatgctgcccagtttgccacggccgatcctgagccacctttggaagagctgggctaccacatctactccagcgacccaccttttgaagttcgtggtgccaatcagtggatcaagtttaagtcagtcagttaaggggaggagaaggagaggttataccttcagggggctaccagacagtgttctcaacttggttaaggaggaagaaaacccagtcaatgaaattcaatgaaattcttggaaacttccattaagtgtgtagattgagcaggtagtaattgcatgcagtttgtacattagtgcattaaaagatgaattattgagtgcttaaagattatttttgacttaaaatagtatactttgaacaaatactctaattatgaaaaggaagaacaattccttgtatgcctgtttcccctgcccccagccacctttttgggaggagaccattatggcggggcccctcacagcattctaccaaccatagcacccaccccgagcagcgctggtgctgcagcctgttcgcgctgaccatttctctacaagatacaatatttattatcaggcaagaggacagttccattttaaaataagacttttgtaatcattccaattttgtaatcatttcaaaggccacataacttagttttctctacttacacattcagtataaatatgaagctattttctgttcatatcaaacattaactacaaggcacattcgtatcagttttgtgtttctcaaattgaagtaccataccagttctgaggcagtgtcccagcttccatgtttgttaaataccccttgtttgtttcaccattccagcaagtgctgaagggtgtactttttttgagacagggtcgggctctgttgcccaggctggagtgcagtggtgtgatcatggctcactgcagcctccacacctcctgggctcaagcaatcctcccacctcagcctcctgcatagctgggactacaagtgaatttcctaatattccgggaggtcaaaaccaaggctcactgttttcacaatacacacagttctatgtttataaataacaggtttcaaaagaaactcaggacagtatttaaaacaagttcttaaactattaattgaacaatggcatttttaaatatgtaaacacagcggaattcgtgtatacactaacagaagctttaacaaaacatgtagcgtggtgggacactctgccacagcttagctgattggtatcaagccttgtctttggtttctgaggcctcctgagcccttctgtactgggagaccgcactccagagtctgcagaggagaccacccctgggaaacaaacacagctgtcttcagagtcagtgcttcaagccaacagagcttaaaactgcagtccctaatttaaaaacctaatgaaaataaaaacattctcctcacatatggaggtgacgctcgtgtcccagcagtagtaggacatggccttagaggtacgtacctgcagagagctggctatttcaaatgactcgggaacaagaaggcaggctgcagtttaaagaagggggtgggtccagcgtgcaggcacgcttgccatgtgcctccacccactcccagccaggcattaatggcaggagattggccagctcttctctgtcacattcctatttctgacttctgcctggctttcagtttctgccccaccttggctttttcccagcttgaacctaatagaactccagagtttggggggaggcccagccctttgttttctgctcttgaagcatattcacacataaaaagttgtattctcttatacaaactgttttgaggctcttaccgtagtcgaaggtatcttagatcttccttagtgatctcattaagaatatccgaaagtgtataaccctcttcaacaatctgaaacaaagatcagatccttaagagctgagcagctgtgtaacaacagcataagaatttctttgttgtaaatttaccttttcaattgtctttgcatcagctccttgcagccgcaaccagtctataagctctttatctgttctctgcccgtaggggcctgctgggttctctgtaatacctgtaacgattggcaatttgttatatattagtctaaccataaaactcttcaaaagtaaccagttggattaataaatgattccagaatgtaaaaaaaaaaaaaaaaa

SEQ ID NO: 14 - DNA, 2338 bp, Homo sapiens
  GenBank ID NM_032782
cgggtgtgctgagctagcactcagtgggggcggctactgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggactaggtctaccttgatcttgaagattcccttggaatgttgagatttaggcttatttgagcactacctgcccaactgtcagtgccagtgcatagcccttcttttgtctcccttatgaagactgccctgcagggctgagatgtggcaggagctcccagggaaaaaggaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgttaaaaaaaaaaaaaaaaaa

SEQ ID NO: 15 - DNA, 3237 bp, Homo sapiens
  GenBank ID NM_013277
gcgaagtgaagggtggcccaggtggggccaggctgactgaatgtatctcctagctatggactaaataatacatggggggaaataaacaagtattcatgagggtgaaaatgtgacccagcaggaaaattacaactattttcaattgacgttgaataggatgagtcatggaatttaagtgatttactgaagattatactactggtagatagaagagctaaagaaagatggatactatgatgctgaatgtgcggaatctgtttgagcagcttgtgcgccgggtggagattctcagtgaaggaaatgaagtccaatttatccagttggcgaaggactttgaggatttccgtaaaaagtggcagaggactgaccatgagctggggaaatacaaggatcttttgatgaaagcagagactgagcgaagtgctctggatgttaagctgaagcatgcacgtaatcaggtggatgtagagatcaaacggagacagagagctgaggctgactgcgaaaagctggaacgacagattcagctgattcgagagatgctcatgtgtgacacatctggcagcattcaactaagcgaggagcaaaaatcagctctggcttttctcaacagaggccaaccatccagcagcaatgctgggaacaaaagactatcaaccattgatgaatctggttccattttatcagatatcagctttgacaagactgatgaatcactggattgggactcttctttggtgaagactttcaaactgaagaagagagaaaagaggcgctctactagccgacagtttgttgatggtccccctggacctgtaaagaaaactcgttccattggctctgcagtagaccaggggaatgaatccatagttgcaaaaactacagtgactgttcccaatgatggcgggcccatcgaagctgtgtccactattgagactgtgccatattggaccaggagccgaaggaaaacaggtactttacaaccttggaacagtgactccaccctgaacagcaggcagctggagccaagaactgagacagacagtgtgggcacgccacagagtaatggagggatgcgcctgcatgactttgtttctaagacggttattaaacctgaatcctgtgttccatgtggaaagcggataaaatttggcaaattatctctgaagtgtcgagactgtcgtgtggtctctcatccagaatgtcgggaccgctgtccccttccctgcattcctaccctgataggaacacctgtcaagattggagagggaatgctggcagactttgtgtcccagacttctccaatgatcccctccattgttgtgcattgtgtaaatgagattgagcaaagaggtctgactgagacaggcctgtataggatctctggctgtgaccgcacagtaaaagagctgaaagagaaattcctcagagtgaaaactgtacccctcctcagcaaagtggatgatatccatgctatctgtagccttctaaaagactttcttcgaaacctcaaagaacctcttctgacctttcgccttaacagagcctttatggaagcagcagaaatcacagatgaagacaacagcatagctgccatgtaccaagctgttggtgaactgccccaggccaacagggacacattagctttcctcatgattcacttgcagagagtggctcagagtccacatactaaaatggatgttgccaatctggctaaagtctttggccctacaatagtggcccatgctgtgcccaatccagacccagtgacaatgttacaggacatcaagcgtcaacccaaggtggttgagcgcctgctttccttgcctctggagtattggagtcagttcatgatggtggagcaagagaacattgaccccctacatgtcattgaaaactcaaatgccttttcaacaccacagacaccagatattaaagtgagtttactgggacctgtgaccactcctgaacatcagcttctcaagactccttcatctagttccctgtcacagagagtccgttccaccctcaccaagaacactcctagatttgggagcaaaagcaagtctgccactaacctaggacgacaaggcaacttttttgcttctccaatgctcaagtgaagtcacatctgcctgttacttcccagcattgactgactataagaaaggacacatctgtactctgctctgcagcctcctgtactcattactacttttagcattctccaggcttttactcaagtttaattgtgcatgagggttttattaaaactatatatatctccccttccttctcctcaagtcacataatatcagcactttgtgctggtcattgttgggagcttttagatgagacatctttccaggggtagaagggttagtatggaattggttgtgattctttttggggaagggggttattgttcctttggcttaaagccaaatgctgctcatagaatgatctttctctagtttcatttagaactgatttccgtgagacaatgacagaaaccctacctatctgataagattagcttgtctcagggtgggaagtgggagggcagggcaaagaaaggattagaccagaggatttaggatgcctccttctaagaaccagaagttctcattccccattatgaactgagctataatatggagctttcataaaaatgggatgcattgaggacagaactagtgatgggagtatgcgtagctttgatttggatgattaggtctttaatagtgttgagtggcacaaccttgtaaatgtgaaagtacaactcgtatttatctctgatgtgccgctggctgaactttgggttcatttggggtcaaagccagtttttcttttaaaattgaattcattctgatgcttggcccccatacccccaaccttgtccagtggagcccaacttctaaaggtcaatatatcatcctttggcatcccaactaacaataaagagtaggctataagggaagattgtcaatattttgtggtaagaaaagctacagtcattttttctttgcactttggatgctgaaatttttcccatggaacatagccacatctagatagatgtgagctttttcttctgttaaaattattcttaatgtctgtaaaaacgattttcttctgtagaatgtttgacttcgtattgacccttatctgtaaaacacctatttgggataatatttggaaaaaaagtaaatagctttttcaaaatgaaaaaaaaaa

SEQ ID NO: 16 - DNA, 18815 bp, Homo sapiens
  GenBank ID NM_001620
agacccgcccgcccgagccggagttacaagagccgcctccgcgcacgggggcccggccactcggagctgctctgccgcggggactgcaccgcccgccctgccagacccgcccggaacggggctcgtcgccgccagtagccgcagcaccgcagccttgggcctcgcgccggctatggccgtgccctggggctgagccctcaggttgtgaccgagattcccgacgagagagactgaggggaagagaggaaggaggggcgggctcctggcaaggcattcgctcctgagcggaatcctgcaaagatggagaaggaggagacaacccgggagctgctgctgcccaactggcagggtagtggctcccacgggctgaccatcgcccagagggacgacggcgtctttgtgcaggaggtgacgcagaactcccctgcggcccgcactggggtggtcaaggagggggaccagattgtgggtgccaccatctactttgacaacctgcagtcgggtgaggtgacccagctgctgaacaccatggggcaccacacggtgggcctgaagctgcaccgcaagggggaccgctctcccgagcctggccagacctggacccgtgaagtcttcagctcctgcagctctgaagtggttctgagcggggatgatgaggagtaccagcgcatctacaccacgaagatcaagccacggctgaagtcggaagatggagtggaaggagacctcggggagacccagagccgtaccatcacagtgaccagaagggtcacggcctacactgtggatgtgactggccgggaaggagccaaggacatagacatcagtagccctgaattcaagatcaagattccaagacatgaactgactgaaatctccaatgtggatgtggagacccagtctgggaagaccgtgatcagactgccctcgggctcgggggcagcctctccgacaggctctgctgtggatatccgagcaggggccatttctgcttcaggaccagagctccaaggtgctggccactcgaagctccaggtcaccatgcctgggataaaggtgggaggctcaggtgtcaatgtcaatgcaaagggcttggacttgggtggcagaggaggggtccaagttccagcagtggacatttcatcttctcttgggggtagggcagtagaggtacagggcccatctctggagagtggtgatcatggcaaaattaaatttcccaccatgaaagtgccgaaatttggtgtctcaacagggcgtgagggccagacaccaaaggcagggctgagggtttctgcacctgaagtctctgtggggcacaagggcggcaagccaggcttgactatccaagcccctcagctggaagtcagtgtgccctctgccaatattgagggccttgaggggaagctgaagggcccccaaatcactgggccatcacttgagggtgacctaggcctgaaaggtgccaagccacaggggcacattggggtggatgcctctgctccccaaattgggggtagcatcactggccccagtgtggaagttcaggcccctgacattgatgttcaggggcctgggagcaaactgaatgtgcccaagatgaaagtccccaagttctctgtatcaggtgcaaagggagaggaaactgggattgatgtgacactgcctacaggtgaagtgactgttcctggggtctctggggatgtcagcctgcctgagattgctactggtgggctggaaggaaagatgaaaggtactaaagtgaagactcctgaaatgattattcagaaacctaaaatctccatgcaggatgtggatctgagccttgggtctcctaaactgaaaggagatattaaggtttctgctcctggggtgcaaggtgatgttaaaggccctcaagtggcacttaaaggctccagagtggacatagagacaccaaacctagagggaaccttgacaggccctaggcttggcagtccttccgggaaaaccggaacctgtaggatctctatgtcagaagtagacttaaatgtggccgcacctaaagtgaaagggggtgtagatgtcacactccccagagtagaagggaaagtcaaagtccctgaagttgatgtcagaggccccaaagtggatgtcagtgccccagatgtcgaagcgcatggcccagaatggaacctgaaaatgcccaagatgaaaatgcccacgttcagcactccaggagccaaaggggaaggtccagatgttcatatgactctacccaaaggagatatcagtatttcagggcccaaggtcaatgtggaagccccagatgtcaacttggagggtctggggggaaaacttaaaggccccgatgttaagctgcctgatatgagtgtcaagacaccaaagatctccatgcctgatgtagatttgcacgtgaaaggtacaaaggtgaagggagagtatgatgtaactgtaccaaagctggaaggagaactcaaaggcccaaaagtggacattgatgccccagatgtggatgttcatggcccagactggcacttgaagatgcccaagatgaaaatgcccaaattcagtgtgccagggttcaaagcagagggcccagaagtggatgtgaacctgcccaaggctgatgtggacatttccgggcccaagatagatgttactgctcctgatgtgagcattgaggaaccagaagggaaattgaaagggcccaagtttaagatgcctgagatgaacatcaaagtccccaagatctccatgcctgatgtggacttacatctgaaaggccctaacgtaaagggagaatatgatgtcacaatgccaaaggttgaaagtgagattaaagttcctgatgttgaacttaaaagtgccaaaatggacattgatgtcccagatgtggaggttcaaggcccagactggcacctgaagatgcccaagatgaaaatgcccaagttcagcatgcctggcttcaaagcagagggcccagaagtggatgtgaacctgcccaaggctgatgtggacatctcaggacccaaggtgggtgttgaagttccagatgtgaatattgaaggacctgaaggaaagctgaagggccccaagttcaagatgccagagatgaatatcaaggcccccaagatctccatgcctgatgtggacttgcatatgaaaggtcctaaagtaaagggagaatatgatatgacagtgccaaagctggaaggggacctgaaaggcccaaaagtagatgtcagtgccccagatgttgaaatgcagggtcctgactggaacttgaagatgccaaagattaaaatgcccaaatttagcatgcccagcctcaaaggagaggggccagaatttgatgtgaacctgtccaaagcgaatgtggacatttctgcaccaaaagtagatactaatgctccagatctgagccttgaaggacctgaagggaagttgaaaggcccgaagtttaagatgcctgagatgcacttcagagctcctaagatgtctttgccagatgttgacctggatcttaaaggacccaaaatgaaaggaaatgtagatatctctgcaccaaagatagagggtgaaatgcaggttccagatgtggacatcagaggtcccaaggtagatattaaagcaccagatgtggaaggccaaggcctggactggagcctgaaaatacccaagatgaaaatgcccaagttcagcatgcccagcctcaaaggcgagggcccagaagtggatgtgaacttgcctaaggctgacgttgttgtctcaggacccaaggtggacatcgaagccccagatgtgagcctcgaaggtccagaagggaagctgaagggtcccaagtttaagatgcctgagatgcatttcaagacccccaagatctccatgcctgatgtggacttacacttgaaaggccccaaagtcaaaggggatgtggatgtgtctgtgcccaaggtagaaggtgaaatgaaagtgccagatgttgaaatcaaaggacccaaaatggacattgatgccccagatgtggaggttcaaggcccagactggcacctgaagatgcccaagatgaaaatgcccaagtttagcatgcctggcttcaaaggagagggccgagaagtggatgtgaacctgcccaaggctgacattgatgtctcaggacccaaggtggatgttgaagtcccagatgtgagccttgagggcccggaaggaaagctgaagggccccaagtttaagatgcctgagatgcacttcaaggcccccaagatctccatgcctgatgtggacctgaatcttaaggggccaaaattgaagggagatgtggatgtgtccttgcctgaggtagaaggtgaaatgaaagtgccagatgttgacattaaagggcccaaagttgacattagtgctccagatgtggatgttcatggcccagattggcacctgaagatgcccaaggtgaaaatgcccaagttcagcatgcccggcttcaaaggagagggccctgaagtggatgtgaagctgcccaaagctgacgttgatgtctcaggacccaaaatggatgctgaagttccagatgtgaatattgaaggtccagacgcaaaactaaaaggtcccaaattcaagatgccagaaatgagtataaagcctcagaagatatccataccagatgttggtttgcatttgaaaggtcctaaaatgaaaggagattatgatgtaacagttccaaaagtagaaggagagataaaagctcctgatgttgacatcaaaggccccaaagttgatattaatgcaccagatgtggaggttcatggcccagactggcacctgaagatgcccaaggtaaaaatgcccaagttcagcatgcctggctttaaaggagagggcccagaggtggatatgaacctgcccaaggctgaccttggtgtttcaggacccaaggtggacattgatgttccagatgtgaatcttgaagctccagaggggaaactaaaaggccctaagttcaagatgccaagcatgaatatacagacgcacaaaatctctatgcctgatgttggacttaatttgaaagcccctaaactgaaaactgatgtagatgtttcccttcccaaagtggaaggagacttgaagggtcctgaaattgatgtgaaagcccctaagatggatgtgaatgttggtgatattgatattgaaggtccagaagggaagttgaagggccccaagtttaagatgcctgagatgcatttcaaggcccccaagatctccatgcccgatgtggacttacacttgaaaggccccaaagtcaaaggggatatggatgtgtctgtgcccaaggtagaaggtgaaatgaaagtgccagatgttgacattaaagggcccaaagtggacattgatgccccagatgtggaggttcacgacccagattggcacctgaaaatgcccaagatgaaaatgcccaagttcagtatgcctggcttcaaagcagagggccctgaagtggatgtgaatctgccaaaggctgacattgatgtgtctggacccagtgtggacactgatgctcctgatttggatattgagggaccagaaggaaagttgaaaggctccaaatttaagatgcccaagttgaatataaaagctcccaaggtctccatgccagatgtggacttgaatttgaagggacccaaactgaagggagagatagatgcttctgtgccagaactggaaggtgatctcagagggccgcaagttgatgtcaaaggtccttttgtggaagcggaggtgcccgatgttgatctggagtgtcctgatgcaaagttgaaagggcccaagtttaagatgcctgagatgcacttcaaggcccccaagatctccatgcctgatgtggacttacacctgaaaggccccaaagtcaaaggggatgcggatgtgtcggtgccaaaattggagggagatttaacaggccccagtgtgggtgtggaggtgcctgatgttgagctggagtgtcctgatgcaaagttgaaaggccctaaatttaagatgccagacatgcacttcaaggcccccaagatctccatgcctgatgtggacttacacttgaaaggccccaaagtcaaaggggatgtggatgtgtcggtgccaaaattggagggagatttaacaggtcccagtgtgggtgtggaggtgcctgatgttgagctggagtgtcctgatgcaaagttgaaagggcccaagtttaagatgcctgagatgcacttcaagacccccaagatctccatgcctgatgtggacttacacctgaaaggccccaaagtcaaaggggatatggatgtgtctgtgcccaaggtagaaggtgaaatgaaagtgccagatgttgacatcaaaggacccaaaatggacattgatgccccagatgtggatgttcatggcccagactggcacctgaagatgcccaagatgaaaatgcccaagttcagcatgcctggcttcaaagcagagggcccagaagtggatgtgaacttgcccaaggctgatgttgttgtctcaggacccaaggtggatgttgaagtcccagatgtgagccttgaaggtccagaagggaagctgaagggccccaagcttaagatgcctgagatgcacttcaaggcccccaagatctccatgcctgatgtggacttacacttgaaaggccccaaagtcaaaggggatgtggatgtgtctttgccaaaattggagggagatttaacaggccccagtgtggatgtggaggtgcctgatgttgagctggagtgtcctgatgcaaagttgaaagggcccaagtttaagatgcctgagatgcacttcaagacccccaagatctccatgcctgatgtgaacttaaacttgaaaggccccaaagtcaaaggggatatggatgtgtctgttcccaaggtagaaggtgaaatgaaagtgccagatgttgacatcagagggcccaaagtggacattgatgccccagatgtggatgttcatggcccagactggcacctgaagatgcctaagatgaaaatgcccaagttcagcatgcctggcttcaaaggagagggcccagaagtggatgtgaacttgcccaaggctgacgttgatgtctcaggacccaaggtggatgttgaagtcccagatgtgagccttgaaggtccagaagggaagctgaagggccccaagtttaagatgcctgagatgcacttcaagacccccaagatctccatgcctgatgttgatttcaatttaaagggacccaaaatcaaaggagatgttgatgtttctgccccaaagctggagggagagttaaaaggtccagaattggatgtcaaaggtcccaaattagatgctgacatgccagaagtagctgtggaaggcccaaatggcaagtggaaaactcctaagttcaagatgccagatatgcactttaaagctcccaaaatctctatgccagacctcgatctacacttgaagagccccaaggcaaaaggagaggtggatgtagatgttcccaaattggaaggggaccttaaagggccacatgtggatgtcagtgggccagacattgacattgagggaccagagggcaaattgaaaggccctaagttcaagatgcctgatatgcatttcaaagcccccaatatttctatgcctgatgttgatctaaatctcaaaggacccaaaatcaagggggatgtggatgtgtctgtgcctgaggtagaaggtaaacttgaagtaccagatatgaacatcaggggccccaaagttgatgtaaatgcccccgatgtccaagctccagactggcacctgaagatgcccaagatgaaaatgcccaagttcagcatgcctggcttcaaagcagagggccctgaagtagacgtcaacttgcctaaggctgacgttgacatctcaggacccaaggtggacattgaaggccctgatgttaatattgaaggaccagagggaaagttgaaagggcctaagttaaagatgccagagatgaacatcaaagcccccaagatctccatgcctgactttgatttgcatctgaaaggtcccaaggtgaagggcgatgtggatgtttctctgcccaaagtggaaggtgacctcaagggccccgaagttgacatcaaggggcccaaagtggatattaatgccccagatgtgggtgttcaaggcccagactggcacctgaagatgcccaaggtgaaaatgccaaagttcagcatgcctggcttcaaaggagagggcccagatggggatgtgaagctgcccaaggctgacattgatgtctcaggacccaaagtggacattgaaggccctgatgttaacattgaaggaccagagggaaagttgaaagggcctaagttcaagatgccagagatgaatatcaaagcccccaagatctccatgcctgatattgacttaaacctgaaaggacccaaagtgaagggtgatgtggatgtttcccttcctaaagtggaaggtgacctcaagggcccagaagttgacatcaagggcccaaaagtggacattgacgcacctgatgttgatgttcatggcccagactggcacctaaagatgcccaagataaaaatgcccaagatcagcatgcctggcttcaaaggagaaggtccagatgtggacgtgaacctgcccaaggctgacattgatgtctcaggaccgaaagtggatgttgaatgtcccgatgtgaatatcgaaggacctgaaggaaagtggaaaagtccaaagtttaagatgccagagatgcattttaagactccaaagatatccatgccagatattgacctgaatctcacaggtccaaaaataaaaggagatgtggatgttacaggccctaaggtagagggagatctgaaaggtcctgaagttgacctcaaaggccccaaagtggacattgatgtcccagatgttaatgttcagggtccagactggcacctgaagatgcccaagatgaaaatgcccaagttcagcatgcctggcttcaaagcagagggccctgaagtggatgtgaacctgcccaaggctgacgttgatgtctcaggccccaaagtggacgttgaaggccctgatgttaacattgaaggaccagagggaaagttgaaagggcccaagttcaagatgccagagatgaatatcaaagcccccaagatccccatgcctgactttgatttgcatctgaaaggtcccaaggtgaagggcgatgtggatatttctctgcccaaagtggaaggtgacctcaagggccctgaagttgacatcaggggtccccaagtggacattgatgtcccggatgtgggcgttcaaggcccagactggcacctaaaaatgcccaaagtgaaaatgcccaaattcagcatgcctggcttcaaaggagagggcccagatgtggatgtgaacctgcccaaggctgaccttgatgtctcaggacccaaggtggacattgatgttccagatgtgaatatcgaaggcccagagggaaagttgaaaggtcccaaattcaaaatgcctgagatgaacatcaaagcccccaagatctccatgcctgacattgatcttaacctgaaaggtcccaaagtgaagggtgacatggatgtgtctctgccaaaagtggaaggtgacatgaaagttcctgacgtggatattaaaggccccaaagtggatattaatgccccagatgtggatgttcaaggcccagactggcacctgaagatgcctaaaataaaaatgcccaagatcagcatgcctggcttcaaaggagaaggtccagaagtggacgtgaacctgcccaaggctgaccttgacgtctcaggacccaaggtggacgttgatgttccagatgtgaatattgaaggtccagatgcgaaactgaagggccctaaattcaagatgccagagatgaacatcaaagctcctaaaatatcaatgcctgatttggacctcaatcttaaaggccctaaaatgaaaggagaggtggatgtttcacttgcaaatgtagaaggtgatttgaaaggacctgctcttgacataaaaggcccaaagatagatgtagatgctccagatattgacattcatggcccagatgccaaattaaaaggtccaaaactgaagatgcctgacatgcatgtaaacatgcccaagatctccatgccagaaattgacttgaatttgaaaggctcaaagcttaagggagatgttgatgtctctgggcccaagttggaaggtgacattaaagctcccagtttggatataaagggcccagaagtggacgtttccggtcctaagcttaatatcgaaggcaagtcaaagaaatctcgttttaagcttcccaaatttaatttttcgggctctaaagttcagacacctgaagtggatgtcaaaggtaaaaagccagatattgacataacaggtccaaaagttgatattaatgctcctgatgtcgaggtccaaggaaaagtgaaaggatccaagtttaaaatgcctttcctgagtatttcatctcccaaagtttctatgcctgacgtggagctaaatttgaaaagtcccaaagtcaaaggagacttagatattgcaggtcccaatttagaaggtgactttaaaggccccaaagtggatattaaggcaccagaagtcaatcttaatgcacctgatgtggatgttcatggtccagactggaatctgaaaatgcccaagatgaaaatgcccaaattcagtgtgtctggcttaaaagcagaagggccagatgtagctgtggatctaccaaaaggagacatcaacatagagggcccaagtatgaacattgagggcccagatctcaatgtggaaggtccggagggaggcttgaaaggtcccaaattcaagatgcctgacatgaatatcaaagctcccaagatctccatgcctgacattgacttaaacttgaaaggccccaaggtgaaaggtgatgtggatatttctcttcccaaacttgaaggggatctgaaagggccagaggttgatatcaaaggccctaaagtggacatcaatgccccagatgtggatgttcatggtccagactggcatctgaagatgcccaaagtgaaaatgcccaagttcagcatgcctggcttcaaaggagaaggccctgaagtcgatgttaccctccctaaagctgacattgacatttctggtcccaatgtagacgttgatgttccagacgtgaatattgaaggtccagatgcaaagctgaagggccccaagttcaagatgcctgagatgaacatcaaagcccccaagatctccatgcctgactttgacctgaacttgaagggacccaaaatgaagggtgatgtggttgtgtctttgcccaaagtggaaggtgatctaaaaggccctgaggtggacatcaagggccccaaagtggacattgacactcctgacattaacatcgaaggctcagagggtaaattcaagggacccaaatttaagataccagagatgcacctgaaggctcccaaaatatcgatgcctgacattgatttaaacctgaagggccccaaagtcaagggcgatgtggatgtttctctgcccaaaatggaaggtgacctcaagggtcctgaagttgacatcaagggccccaaagtggacattaatgctccagatgttgatgttcaaggcccagactggcacctgaagatgcccaaggtgaaaatgcccaagttcagcatgcctggcttcaaaggagagggcccagatgtggatgtgaacctgcccaaggctgaccttgatgtctcaggacccaaggtggacattgatgttccagatgtgaatatcgaaggcccagagggaaagttgaaaggtcccaaattcaagatgcctgagatgaacatcaaagcccccaagatctccatgcctgacattgatcttaacctgaaaggacccaaagtgaagggtgatatggatgtgtctctgccaaaagtggaaggtgacatgcaagttcctgacttggatattaaaggccccaaagtggatattaatgccccagatgtggatgttcgaggcccagactggcacctgaagatgcctaagataaaaatgcccaagatcagcatgcctggcttcaaaggagaaggtccagaagtggatgtgaacctgcccaaggctgaccttgacgtctcaggacccaaggtggacgttgatgttccagatgtgaatattgaaggtccagatgcgaaactgaagggccctaaattcaagatgccagagatgaacatcaaagcccccaagatctccatgcctgactttgatttgcatctgaaaggccctaaggtgaaaggagatgtggatgtttctctgcctaagatggaaggtgatctaaaggcccctgaagttgacatcaagggccccaaagtggacattgatgccccagatgtggatgttcatggcccagactggcacctgaagatgcccaaggtgaaaatgcccaaattcagcatgccaggatttaaaggagagggcccagaagtggatgttaatttgcccaaagctgacattgatgtctcaggacccaaagtggacattgacactcctgatattgatattcatggtccagaagggaaactgaagggccccaaatttaaaatgcctgacctgcacctcaaggcaccgaagatctctatgcctgaagttgacctgaatctgaaaggtccaaagatgaagggcgacgtggacgtttctctgcccaaagtggaaggcgacctcaagggccctgaagttgacatcaagggccccaaagtggacattgatgtcccagatgtggacgttcaaggcccagactggcacttaaaaatgcccaaagtgaaaatgcccaagttcagcatgcctggcttcaaaggagagggcccagatgtggatgtgaacctgcccaaggctgaccttgacgtctcaggacccaaggtggacattgatgttcctgatgtgaatatcgaaggtccagatgcgaaactaaagggccctaaattcaagatgcctgagatgaacatcaaagcccccaagatctccatgcctgactttgatttgcatctgaaaggtcccaaggtgaagggtgatgtggatgtttcccttcctaaagtggaaggtgacctcaagggcccagaagttgacatcaagggccccaaagtggacatcgatgcccctgatgtagatgttcatggcccagactggcacctgaagatgcccaaggtgaaaatgcccaaattcagcatgccaggattcaaaggagagggcccagatgtggatgttacccttcctaaggctgacattgagatttctggccccaaagtggacattgatgcccctgatgtcagtatcgaaggtccagatgcaaaactcaagggtccaaagttcaagatgccagagatgaacatcaaggcccccaaaatctccatgcctgacattgactttaacttgaagggtcccaaagtgaaaggtgatgtggatgtctctctgcccaaagtggaaggtgatctcaagggccctgaaattgacataaaaggccccagtttggacattgacacacctgatgtcaatattgaaggtccggaaggaaaattgaaggggcccaaatttaagatgcctgagatgaacatcaaagctcccaaaatctctatgcctgactttgatttgcacctgaaaggtcccaaggtgaagggtgatgtggatgtttcactacctaaggtggaaagtgatctgaaagggccagaggtagacattgaaggtcctgaagggaagctcaaaggtcccaagtttaagatgcctgatgtacatttcaaaagcccacaaatctccatgagtgacattgatttgaatttgaaaggacctaagataaaaggagatatggacatttccgttcctaaactggagggagatctgaaaggtcccaaagtggatgtcaaaggccctaaagtgggcattgacactcctgatattgacattcatggtccagaagggaaactgaagggccccaaatttaaaatgcctgacttacacctcaaggcaccgaagatctctatgcctgaagttgacctgaatctgaaaggtccaaaggtgaagggcgacatggacatttctctgcccaaagtggaaggcgacctcaagggccccgaagttgacatcagggaccccaaagtggacattgatgtcccagatgtggacgttcaaggcccagactggcacctaaaaatgcccaaagtgaaaatgcccaagttcagcatgcctggcttcaaaggagagggcccagatgtggatgtgaacctgcccaaggctgacattgatgtctcaggacccaaagtggacgttgatgttcctgatgtgaatatcgaaggtccagatgcgaaactaaagggccccaagttcaagatgcctgagatgagcatcaaagcccccaagatctccatgcctgatattgacttaaacctgaaaggacccaaagtgaagggcgatgtggatgttacccttcctaaagtggaaggtgacctcaagggcccagaagctgacatcaagggcccaaaagtggacatcaacacccctgatgtggatgttcatggcccagactggcacctgaagatgcccaaggtgaaaatgcccaaattcagcatgcctggcttcaaaggagaaggtccagatgtggatgtgagcctgcccaaggccgacatcgatgtctcgggacccaaggtggacgttgatattccagatgtgaatatcgaaggtccagacgcaaaactgaagggccccaagttcaagatgcctgaaataaatatcaaagctcccaagatctccatacctgatgttgacctggatttgaaaggacccaaagtaaaaggagattttgatgtgtctgtccctaaggttgaagggactttgaaaggcccagaagtagatcttaaaggtccacgtctggatttcgaaggccctgatgccaaactcagtggcccatctttgaagatgccatcgctggagatatctgctcctaaagtaactgctcctgatgttgatttgcatctcaaggcaccaaaaattggattttcaggtccgaagttagaaggtggtgaagtggacctcaagggacccaaagttgaagctccaagcttagatgtacacatggacagcccagatattaacatcgaagggccagatgttaaaatccccaaatttaagaaacccaagtttggatttggggcaaaaagccccaaagctgacatcaagtcaccttcactggatgtcactgttcctgaggcagagctgaaccttgagactcctgaaattagtgttggtggcaagggcaagaaaagtaagtttaaaatgcctaaaattcatatgagtggtcctaagattaaggccaaaaaacagggatttgacctgaatgttcctgggggtgaaattgatgccagcctcaaggctccggatgtagatgtcaacatcgcagggccggatgctgcactcaaagtcgacgtgaaatcgcccaaaaccaagaaaacgatgtttggaaaaatgtacttcccagatgtagagtttgacattaaatcacctaaatttaaagctgaggcccctctccctagccccaaactggagggtgaactccaggcacctgatctggaactttctttgccagcgattcacgtcgaaggtcttgacatcaaggcgaaggctcccaaggtcaagatgccagatgtggacatctcagtgccaaaaatagagggtgacctgaaaggccccaaagtgcaggcaaacttgggtgcacctgacatcaacatcgaaggcctagatgctaaagtcaaaacaccgtccttcggcatttctgcccctcaagtctccatccctgatgtgaatgtaaacttgaaaggaccaaagataaagggtgatgtccccagcgtgggactggaaggaccagatgtagatctgcaaggtccagaagcaaaaattaagttccccaagttttccatgcccaagatcggcatcccaggtgtgaaaatggagggtgggggagccgaggtccatgcccagctaccctctcttgaaggagacttgagaggaccagatgttaagctcgaagggcccgatgtttctctaaaggggccaggagtagacttgccttcagtgaacctctctatgccaaaagtctctgggcctgaccttgatctgaacttgaaaggaccaagtttgaagggagacctggatgcatctgttcccagcatgaaggtgcatgctccagggctcaacctcagtggtgtcggtggcaaaatgcaggtgggaggagacggtgtgaaagtgccagggatcgatgccacaacaaagcttaacgttggggcaccagatgtgacactgaggggaccaagcctgcagggagatctggctgtctctggtgacatcaaatgccctaaagtatccgtaggagctcctgatctaagcttggaggcatccgaaggcagcattaaacttcccaaaatgaagctgccccaatttggcatctctactccggggtccgacttgcacgtcaatgccaaggggccacaggtttctggcgaactgaaggggccaggtgtggatgtgaacctgaaagggcctcggatttcagcaccgaatgtggactttaacttggaaggaccaaaagtgaaagggagccttggggccactggtgagatcaaaggccccactgtcggaggaggtcttccaggcattggtgttcaaggcctagaaggaaacctccagatgcctggaattaagtcctctggatgtgatgtgaacctgccaggcgtgaatgtgaaactcccaactgggcagatttctgggcctgaaatcaaaggtggtctgaaaggttcagaagtaggtttccatggggctgctcctgatatcagtgtgaaggggcctgcctttaatatggcatctcctgagtcagattttggcatcaacttgaagggcccaaaaatcaaaggaggtgcggatgtttcagggggtgtcagtgccccagacatcagccttggtgaagggcatttgagtgttaaaggttccgggggtgagtggaagggaccccaagtctcctctgctctcaacttggacacatctaagtttgctgggggccttcatttctcaggaccaaaggtggaaggaggtgtgaaaggaggtcagattggactccaggctcctgggctgagtgtgtctgggcctcaaggtcacttggaaagtggatctggaaaagtaacattccctaaaatgaagatccccaaatttaccttctctggccgtgagctggttggcagagaaatgggggtggatgttcacttccctaaagcagaggccagcatccaagctggtgctggagacggcgagtgggaagagtctgaagtcaaactgaaaaagtccaagatcaaaatgcccaagtttaatttttccaaacctaaagggaaaggtggtgtcactggctcaccagaagcatcaatttctgggtccaaaggtgacctgaaaagttcaaaggccagcctgggctctctggaaggagaggcagaggccgaagcctcttcaccgaaaggcaaattctccttatttaaaagtaagaagccacggcaccgctcaaattcattcagtgatgaaagagagttctctggaccttccaccccgacggggacgctggagtttgaaggtggggaagtgtctctggaaggtgggaaagttaaagggaaacacgggaagctgaaattcggtacctttggtggattggggtcaaagagcaaaggtcattatgaggtgactgggagcgatgatgagacaggcaagttacaggggagtggggtgtccctggcctctaagaagtcccgactgtcctcctcttctagcaatgacagtgggaataaggttggcatccagcttcccgaggtggagctgtcagtttccacaaagaaagagtagcaggcctttgtatgtgtgtacatatatatatatataacaaaacatcagccttgggtggtgtgttcctatataaactccaaagggaaacacaccgactgcctcagcaatcatgcaaagaccttgcctggcccggtggcaagcgctgaaaaaccgaccgcctgtaggctcctggaactatacagataggtaaagagttccaagttcgtccagcccatgtgcaaagtcaacagtatttgccttaagatttcatatatatatatttttttgcattgactgctgagagctcctgtttactaagcaagcttttgtgtttattatcctcatttttactgaacattgttagttttggggtaatggaaacccactttttcattgtaatgactttgggggcttttgttagtaagggtgggtggggtgatgggttgcagacggaggtcaggtcttcctctttcctgagactggatctgttcaaacagcaaacgcccacagatggcccagaggtggtggtagtcagggtgtgtgggtgtttttagggttctttagtgttgtttctttcacccaggggtggtggtcccagccagtttggtgctgacggtgagaggaaattagaatctgtttgcaaattgtccaacccaccccctcaacatgaggggcttccattttctgtgttttgtaagggaactgtttccttcatgccgccatgttcctgatattagttctgatttctttttaacaaatgttatcatgattaagaaaatttccagcactttaatggccaattaactgagaatgtaagaaaattgatgctgtacaaggcaaataaagctgtttattaaccttga

SEQ ID NO: 17 - DNA, 2194 bp, Homo sapiens
  GenBank ID NM_030760
ggcagcgcgactgcgggtggcgcacgaccagggcgcagaccttggggcgcgcggcccatggagtcggggctgctgcggccggcgccggtgagcgaggtcatcgtcctgcattacaactacaccggcaagctccgcggtgcgcgctaccagccgggtgccggcctgcgcgccgacgccgtggtgtgcctggcggtgtgcgccttcatcgtgctagagaatctagccgtgttgttggtgctcggacgccacccgcgcttccacgctcccatgttcctgctcctgggcagcctcacgttgtcggatctgctggcaggcgccgcctacgccgccaacatcctactgtcggggccgctcacgctgaaactgtcccccgcgctctggttcgcacgggagggaggcgtcttcgtggcactcactgcgtccgtgctgagcctcctggccatcgcgctggagcgcagcctcaccatggcgcgcagggggcccgcgcccgtctccagtcgggggcgcacgctggcgatggcagccgcggcctggggcgtgtcgctgctcctcgggctcctgccagcgctgggctggaattgcctgggtcgcctggacgcttgctccactgtcttgccgctctacgccaaggcctacgtgctcttctgcgtgctcgccttcgtgggcatcctggccgcgatctgtgcactctacgcgcgcatctactgccaggtacgcgccaacgcgcggcgcctgccggcacggcccgggactgcggggaccacctcgacccgggcgcgtcgcaagccgcgctcgctggccttgctgcgcacgctcagcgtggtgctcctggcctttgtggcatgttggggccccctcttcctgctgctgttgctcgacgtggcgtgcccggcgcgcacctgtcctgtactcctgcaggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcagactgacaccctcggcccacgactgtcttcccaagttttacagacttgttctttttacataaaggaatttgtaggaaatgcagccaaaggtgcagtcggaaaagatgcaggggaaatgtatttatgcagcgacaccccacaatgtgaacaaacagacaaaaaatctgtgccctcgtggaattgacgttctgcttgggaacacagaaaagaactcggtgatgaaataatggagatgattccagtgacaaacgacagagatggtgatggtggtcagggaagacctctctgcagaggtagtgacttgtgatgtgagctgagacctctgtcctgggaagaccaaaagaaaagcatttcaggatgagggaatggcatgcgcaaaggccctgaggctgaaatgtgcccatgtgttctaagaaatgcagcgatgctggtgtgcctggagcagggacggagggggagaatgggaggagacaaggagctgaaggagtagttcccgaaggaccttgtgggtgatatagaggacttcgcttttgctctgagtgaggtgggagccatagaagcttctaagcagaagagggacttgccctaattcaggtgatcacaggtgtcttgtggcctccatgggaggttgaaaaccagagaaggtgaaggggggctgcactgagccacaggaacaatgatggagattccagctaagcccagaccccgtggattctagatagattttagaggcagcagacagaattactgaggaattgagtgtaagagtggaataaagttatcaaggacaatgccaagggtggggcacccccaaatttgactctgggagactcagccaaatcctatctggtaataaaatttcttttttatttttaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 2545 bp, Homo sapiens
  GenBank ID NM_004419
actcattcacataaaacgctgcgcggccggcggaatccccggcttctagggcggcgagcggccgggctggctatcgagcgagcggggcgggaacgcggagttgcgccgccgctcgggcgccgggctccgtcgcggccgcagccccgcgggtcgccctcccgtgcctcgcccgcggacaccctggccgtggacaccctggccgtgggcacccgcggggcgcgcggcgcggggccgctggccggcggcggcggcggcatgaaggtcacgtcgctcgacgggcgccagctgcgcaagatgctccgcaaggaggcggcggcgcgctgcgtggtgctcgactgccggccctatctggccttcgctgcctcgaacgtgcgcggctcgctcaacgtcaacctcaactcggtggtgctgcggcgggcccggggcggcgcggtgtcggcgcgctacgtgctgcccgacgaggcggcgcgcgcgcggctcctgcaggagggcggcggcggcgtcgcggccgtggtggtgctggaccagggcagccgccactggcagaagctgcgagaggagagcgccgcgcgtgtcgtcctcacctcgctactcgcttgcctacccgccggcccgcgggtctacttcctcaaagggggatatgagactttctactcggaatatcctgagtgttgcgtggatgtaaaacccatttcacaagagaagattgagagtgagagagccctcatcagccagtgtggaaaaccagtggtaaatgtcagctacaggccagcttatgaccagggtggcccagttgaaatccttcccttcctctaccttggaagtgcctaccatgcatccaagtgcgagttcctcgccaacctgcacatcacagccctgctgaatgtctcccgacggacctccgaggcctgcgcgacccacctacactacaaatggatccctgtggaagacagccacacggctgacattagctcccactttcaagaagcaatagacttcattgactgtgtcagggaaaagggaggcaaggtcctggtccactgtgaggctgggatctcccgttcacccaccatctgcatggcttaccttatgaagaccaagcagttccgcctgaaggaggccttcgattacatcaagcagaggaggagcatggtctcgcccaactttggcttcatgggccagctcctgcagtacgaatctgagatcctgccctccacgcccaacccccagcctccctcctgccaaggggaggcagcaggctcttcactgataggccatttgcagacactgagccctgacatgcagggtgcctactgcacattccctgcctcggtgctggcaccggtgcctacccactcaacagtctcagagctcagcagaagccctgtggcaacggccacatcctgctaaaactgggatggaggaatcggcccagccccaagagcaactgtgatttttgtttttaagactcatggacatttcatacctgtgcaatactgaagacctcattctgtcatgctgccccagtgagatagtgagtggtcaccaggcttgcaaatgaacttcagacggacctcagggtaggttctcgggactgaaggaaggccaagccattacgggagcacagcatgtgctgactactgtacttccagacccctgccctcttgggactgcccagtccttgcacctcagagttcgccttttcatttcaagcataaggcaataaatacctgcagcaacgtgggagaaagaagttgctggaccaggagaaaaggcagttatgaagccaattcattttgaaggaagcacaatttccaccttattttttgaactttggcagtttcaatgtctgtctctgttgcttcggggcataagctgatcaccgtctagttgggaaagtaaccctacagggtttgtagggacatgatcagcatcctgatttgaaccctgaaatgttgtgtagacaccctcttgggtccaatgaggtagttggttgaagtagcaagatgttggcttttctggattttttttgccatgggttcttcactgaccttggactttggcatgattcttagtcatacttgaacttgtctcattccacctcttctcagagcaactcttcctttgggaaaagagttcttcagatcatagaccaaaaaagtcataccttcgaggtggtagcagtagattccaggaggagaagggtacttgctaggtatcctgggtcagtggcggtgcaaactggtttcctcagctgcctgtccttctgtgtgcttatgtctcttgtgacaattgttttcctccctgcccctggaggttgtcttcaagctgtggacttctgggatttgcagattttgcaacgtggtactactttttttttctttttgtctgttagttatttctccaggggaaaaggcaataattttctaagacccgtgtgaatgtgaagaaaagcagtatgttactggttgttgttgttgttcttgttttttatagtgtaaaataaaaatagtaaaaggagaaaagcaaaaaaaaaaaaaaaaaa